(19) United States
(12) Patent Application Publication
(10) Pub. No.: US 2020/0085877 A1
(43) Pub. Date: Mar. 19, 2020

(54) ELESTOMERIC FIBROUS HYBRID SCAFFOLD FOR IN VITRO AND IN VIVO FORMATION

(71) Applicant: The Children's Medical Center Corporation, Boston, MA (US)

(72) Inventor: Nafiseh Masoumi, Boston, MA (US)

(21) Appl. No.: 16/615,027

(22) PCT Filed: May 21, 2018
(86) PCT No.: PCT/US2018/033736

§ 371 Date: Nov. 19, 2019

Related U.S. Application Data
(60) Provisional application No. 62/508,832, filed on May 19, 2017.

(51) Int. Cl. A61K 35/28; A61K 35/44; A61L 27/18; A61L 27/38; A61L 27/48; A61L 27/50; A61L 27/52; C12N 5/00

(52) U.S. Cl. CPC A61K 35/28; A61K 35/44; A61L 27/18; A61L 27/3834; A61K 2035/124; A61L 27/507; A61L 27/52; C12N 5/0068; A61L 27/48

(57) ABSTRACT

Biocompatible hybrid fibrous scaffold, derived from a synthetic polymer and a natural hydrogel, and methods of use thereof in tissue engineering.

### CLAIM OF PRIORITY

This application claims the benefit of U.S. Provisional Patent Application Ser. No. 62/508,832, filed on May 19, 2017. The entire contents of the foregoing are hereby incorporated by reference.

### TECHNICAL FIELD

Described herein are biocompatible hybrid fibrous scaffolds, derived from a synthetic polymer and a natural hydrogel, and methods of use thereof in tissue engineering.

### BACKGROUND

Synthesis and fabrication of naturally derived biomaterials or synthetic polymers to mimic the structural and mechanical characteristics of native tissues has been the focus of tissue engineering for more than a decade. Modern biomaterials, however, are not without limitations.

### SUMMARY

Described herein is a platform to develop a biocompatible hybrid fibrous scaffold, derived from a synthetic polymer and a natural material, e.g., gelatin or a hydrogel, that facilitates cell attachment, ingrowth and elongation along the fiber direction. Our initial target was to match the constructs' mechanical properties and structure with the native tissue's properties, such as ECM composition and organization, as well as mechanical stiffness and anisotropy. In an attempt to mimic native tissues composition and overcome the challenge of cell ingrowth in fibrous constructs, we used two approaches to fabricate our cell friendly scaffold. First, we have tested several hydrogels as cell carrier to incorporate the cells into a 3D fibrous P4HB construct and provided a ready to implant scaffold. The data we present here includes photocrosslinkable GelMA to encapsulate cells, an approach that resulted in 3D tissue formation throughout the hybrid scaffold. Photodegradable GelMA was shown to be sensitive to UV irradiation; enabling us to manipulate the physical properties and degradation rate of the hydrogel within the scaffolds. Next, we created a P4HB composite trilayered structure with P4HB on the outside and P4HB/Gelatin as middle layer. Combining the two materials offered a cell compatible environment while providing both sufficient mechanical support and structural anisotropy. Our hybrid scaffolds were able to guide the cellular arrangement due to their fiber orientation by providing a dynamic cell culture substrate due to the presence of photodegradable GelMA hydrogels, which resembled native tissue architectures. Our results suggest that the hybrid scaffold can serve as a suitable replacement to address the requirements for cardiovascular tissue engineering. Furthermore, our in vivo evaluations revealed excellent biocompatibility and minimal degradation, resulting in early and progressive ingrowth of host tissue for the hybrid scaffolds, confirming that the material is 1) capable of withstanding physiological pressures on the surface of the pulmonary artery, 2) does not induce clot formation, 3) permits myofibroblast activity across the scaffold, and 4) produces aligned tissue growth on the scaffold surface that is in contact with blood. In vitro testing of these materials as heart valve leaflets in a bioreactor system that mimics the pressure and flow conditions found in the circulation demonstrated durability for up to two weeks and continued viability of cells that were incorporated into the scaffold material.

In some embodiments, the hydrogel is used as a cell carrier to provide the right environment for the cells, and wherein the hydrogel would be degraded in 2-3 days leaving the cells attached to fibers throughout the scaffolds.

In some embodiments, the composite P4HB-Glatin has a physical structure that is a fibrous matrix (e.g., contains layers of align 8-10 um fibers). The material is semi elastic (has a liner stress-strain curve). The composite structure is tri-layered. In some embodiments, the mixture of P4HB-Glatin forms monomers of small chains during the creation of fibers under electrical field.

Provided herein are elastomeric scaffolds for soft tissue engineering comprising a poly-4-hydroxybutyrate (P4HB) matrix. In some embodiments, the scaffolds also comprise a hydrogel, preferably a photocrosslinkable hydrogel, e.g., gelatin or methacrylated gelatin (GelMa).

In some embodiments, the scaffolds comprise a P4HB matrix, wherein the hydrogel is distributed throughout the matrix.

In some embodiments, the scaffolds comprise an inner layer of a gelatin/P4HB composite, and an outer layer of P4HB on either side of the inner layer.

In some embodiments, the scaffolds are fabricated by dry spinning to generate aligned fibers of P4HB.

In some embodiments, the P4HB matrix has an average fiber diameter of 5-20 μm, preferably 8-10 μm, and/or a porosity of 10-15 μm.

In some embodiments, the hydrogel encapsulates a plurality of cells, preferably stem cells, preferably mesenchymal stem cells (MSCs) or Valvular Interestitial Cells. Other cell types can also be used.

In some embodiments, the surface of the scaffold comprises cells, preferably cells of a second cell type, preferably endothelial progenitor cells (EPCs), preferably derived from circulating blood.

Also provided herein are methods for forming an artificial tissue; the methods include culturing a scaffold as described herein in a cyclic stretch/flexure bioreactor or in a bioreactor that delivers flow, flexion, and shear signals to the scaffold.

Further, provided herein are artificial tissues formed by a method described herien, e.g., wherein the tissue is a heart valve leaflet, vascular conduit or blood vessel, or a portion thereof. Blood vessels are typically smaller, while conduits refers to large aortic or pulmonary walls.

In addition, provided herein are methods for of replacing a tissue in a subject, the method comprising implanting into the subject the scaffold of claims 1-5.

Also provided are methods for forming an artificial tissue. The methods include fabricating or providing an elastomeric scaffold comprising poly-4-hydroxybutyrate (P4HB), wherein the scaffold is fabricated, e.g., by dry spinning, to generate aligned fibers of P4HB to form an anisotropic matrix; contacting the elastomeric scaffold with a hydrogel, preferably a photocrosslinkable hydrogel, wherein the hydrogel encapsulates a first plurality of cells, preferably stem cells, preferably mesenchymal stem cells (MSCs), under conditions such that the hydrogel is distributed throughout the scaffold; optionally seeding the surface of the hydrogel-scaffold with a second plurality of cells, preferably cells of a different origin from the first plurality, preferably EPCs, preferably isolated from circulating blood; exposing the cell-seeded scaffold to light sufficient to crosslink the hydrogel; and culturing the scaffold under conditions sufficient to allow proliferation and optionally differentiation of the cells, thereby forming an artificial tissue.

An additional method of forming an artificial tissue include fabricating or providing an elastomeric scaffold comprising a poly-4-hydroxybutyrate (P4HB)/gelatin matrix comprising an inner layer of a gelatin/P4HB composite, and an outer layer of P4HB on either side of the inner layer, wherein the scaffold is fabricated by: generating a first layer of aligned fibers of P4HB; forming a layer comprising a P4HB/gelatin composite on the matrix; and generating a second layer of aligned fibers of P4HB; preferably wherein the gelatin encapsulates a first plurality of cells, preferably stem cells, preferably mesenchymal stem cells (MSCs); optionally seeding the surface of the hydrogel-scaffold with a second plurality of cells, preferably cells of a different origin from the first plurality, preferably EPCs, preferably isolated from circulating blood; exposing the cell-seeded scaffold to light sufficient to crosslink the hydrogel; and culturing the scaffold under conditions sufficient to allow proliferation and optionally differentiation of the cells, optionally comprising culturing a scaffold as described herein a cyclic stretch/flexure bioreactor or in a bioreactor that delivers flow, flexion, and shear signals to the scaffold, thereby forming an artificial tissue.

In some embodiments, the artificial tissue is shaped to be used as a heart valve leaflet, vascular conduit or blood vessel.

In some embodiments, the photocrosslinkable hydrogel is methacrylated gelatin (GelMa).

Further provided herein are methods of replacing a tissue in a subject, comprising implanting into the subject an artificial tissue as described herein, e.g., prepared by a method described herein. In some embodiments, the methods are used for replacing a heart valve leaflet, vascular conduit or blood vessel, or a portion thereof, in a subject, and include implanting into the subject an artificial heart valve leaflet, vascular conduit or blood vessel as described herein, e.g., prepared by a method described herein.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-1H. Physical production and mechanics of P4HB. (A) Schematic and chemical structure of novel, dryspun P4HB material. SEM images of (B) aligned and (C) random P4HB fibers to show variation in fiber arrangement. (D-E) F-actin images comparing cellular alignment and spreading on aligned and random fibers of P4HB scaffolds. Aligned fibers show an increased number of cellular connections due to organized arrangement of cells in the fiber direction. (F) Comparison of fiber and pore sizes for aligned and random fibers within sheets of P4HB. While the fiber sizes remain relatively similar in both types, the pore sizes in aligned sheets are smaller than those of the random sheets, which suggests that it can promote cellular connectivity and communication. The results correspond with the SEM images. (G) Representative of the stress-strain curve for the aligned and random scaffolds confirms the anisotropic mechanical properties of the scaffolds containing aligned fibers. (H) Comparison of the mechanical characteristics of both random and aligned fiber sheets of P4HB.

FIGS. 2A-2D. Cyclic tensile tests among various scaffold materials. (A) Image on far left panel represents the initial position of scaffolds before cyclic tests where the scaffold positioned is held straight between the gauges. The subsequent images represent the deformed position of each material following 5 cycles. (B) Representative of the stress-strain curves for each of the fibrous scaffolds (P4HB, PCL, PCUU/PGS). Similar to elastic PCUU, P4HB showed little-deformation. (C) When sutured, P4HB retained sutures and withstood ultimate tensile stresses to a higher degree than that of sheep pulmonary artery. (D) The elastic modulus of P4HB proved to be comparable to many other cardiac tissues and was most similar to valve leaflets and aortic vessels.

FIGS. 3A-3K. Cellular encapsulation, distribution and viability were examined prior to implantation. (A) Schematic of MSC direct surface seeding onto bare P4HB. (B) Histology showing the cellular distribution on scaffolds. For direct surface seeding, the cells did not penetrate the scaffold and remained primarily attached to the surface layer. (C) Live/Dead assay confirmed the viability of cells on those scaffolds. (D-F) Schematic of the cell encapsulation with GelMa on P4HB. Histology and Dapi analysis confirmed that the cell distribution here is seen throughout the scaffold. SEM images compare the pore and surface variations at Day 1 between (G) bare P4HB and (H) hybrid P4HB/GelMa. The pores are completely filled with cross-linked GelMa compared with the bare P4HB scaffold. SEM images from Day 7 show the pore and surface variations for scaffolds: hybrid with no cells (I), hybrid with encapsulated MSCs (J), and bare P4HB with direct surface seeding (K). Figure J shows cells spread more evenly and some tissue formation, especially when compared to Figure I, in which GelMa degraded and left an uneven, porous surface.

FIGS. 4A-4L. Mechanical properties of hybrid scaffolds with MSCs were compared in both static and bioreactor conditions. (A-B) Schematic representation and actual images of the stretch-flex bioreactor used. (C-D) Image of the scaffold in flexed (C) and stretch (D) states within the bioreactor. (E-G) Comparison of collagen, DNA, and collagen/DNA values determined from both static and bioreactor cultures. Though DNA values were higher in the static samples, the collagen produced per DNA was higher in the bioreactor, suggesting that physical stimulation increased cellular enzymatic activity. H) F-actin confirms the presence and progression of MSCs across the scaffold following bioreactor cultivation. Comparison of hybrid scaffold mechanical properties in both (I) static and (J-L) bioreactor conditions, with and without cells over the 2-week culture time. The presence of cells increased the some mechanical values in hybrid scaffolds placed in both static and bioreactor conditions. Higher stiffness for scaffolds without cells in the bioreactor suggests increased alignment but lower UTS, and higher deformation for these scaffolds is due to the higher rate of degradation under mechanical stimulation. The increased values of stiffness and UTS for the scaffolds with the cells confirmed that scaffolds hold their integrity and ECM generation, which correlated with collagen/DNA data results in improved mechanical properties.

FIGS. 5A-5K. In Vivo experiments assessed the functionality of hybrid scaffolds under physiological pressure and stress. (A) Schematic of the hybrid patch sized, cut, and placed as a patch on sheep pulmonary artery (B-C) Actual images from surgical patch implant, both with sutures, prior to explant (B) and post-explant of patch with native tissue attached (C). Images (D) and (E) portray standard H&E stains of the hybrid scaffold with tissue formation, post-explant, in cross-sectional (D) and surface (E) orientations. (F-G) Magnifications of the H&E stains show presence of cells and sites of potential lumen or pore formation. (H-I) Hybrid scaffolds were also stained for α-SMA to confirm cell integrity and motility. The majority of cells were within the interior of the scaffold. (J-K) Stains for CD45 were performed to confirm the presence of various cell types, if any, but the stain was inconclusive; magnifications (K) suggest possible sites where cells are beginning to line preliminary lumen.

FIG. 6: Dapi staining of the cell nuclei on the P4HB scaffolds with random and aligned fibrous structures. The attachment analysis (measured from the number of cell nuclei stained on the surface of the scaffolds and DNA Pico green assay) detected higher cell numbers in aligned scaffolds versus scaffolds comprise of random fibers.

FIGS. 7A-7D. The pressure test confirmed that P4HB-Gelma scaffolds held 100 mmHg of hydrostatic pressure while fibrous structures leaked due to the porosity in the scaffold.

FIGS. 8A-8E. Bioassays results performed on scaffolds with random fibers also confirmed the results obtained with aligned fibrous scaffolds. Culturing the seeded scaffolds in the stretch/flexure bioreactor resulted in the higher production of ECM (i.e., Collagen and GAG). The results were in accordance with improved mechanical properties of the scaffolds after being cultured in the bioreactor. The higher E and UTS for non-seeded scaffolds in the bioreactor was due to random fibers reorienting toward the stress direction and forming a more aligned fibers.

FIGS. 9A-9D. Mechanical properties of the scaffolds seeded in static cultures for a period of 4 weeks. Lower UTS and E are the clear induication of scaffolds degredation after incubation for a month without cells.

FIG. 10. Thrombogenicity assay showed that hybrid scaffolds can attract blood cells and therefore, scaffolds seeded with endothelial cells (EC) showed no sign of plasma attached to the scaffolds. The EC seeding process was quantified and optimized via staining process.

FIGS. 11A-11B. Stress-strain test of P4HB composite scaffold seeded with EPCs after 72 hours of seeding (11A) and after 4 weeks of culture (11B). The scaffold shows anisotropic properties at both time points. The preferred direction shows a higher ultimate tensile strength (UTS) and strain at the UTS (e) and elasticity (E) in the preferred direction (PD) over the orthogonal direction (XD).

Table for 11A:

| | PD | XD |
|---|---|---|
| UTS | 3320 | 1336.363636 |
| e | 0.3394 | 0.1564 |
| E_{trans.} | 18499 | 9260.9 |

Table for 11B:

| | PD | XD |
|---|---|---|
| UTS | 1173.049645 | 594.3262411 |
| e | 0.513404255 | 0.481702128 |
| E_{trans.} | 3941.2 | 1917.6 |

FIGS. 12A-12B. Representative biaxial stress-strain curves of tri-layered P4HB composite scaffolds presented in PD and XD directions, indicating the anisotropic properties. Further, 80, 90 and 100 μm thick scaffolds show comparable values and the material shows similar behavior as the native leaflet under 0.3 strain.

FIGS. 13A-13B. Cross-section of nanofibers of tri-layered P4HB composite after 10 days in culture shows the three distinctive layers of the scaffold. Scale bar presents 50 μm (13A) and 20 μm (13B).

FIGS. 14A-14B. Ex-vivo test of tri-layer P4HB composite scaffold measured from the PV position in a pig heart shows similar behavior to the native leaflets under pulmonary pressure (about 30 mmHg). The scaffold is able to fully open (14A) and enclose with the native pulmonary valves (14B).

FIGS. 15A-15B. Ex-vivo test of tri-layered P4HB composite scaffold measured from the PV position in a pig heart shows similar behavior to the native leaflets under aortic pressure (about 80 mmHg). The scaffold is able to fully open (15A) and enclose with the native pulmonary valves (15B) without failure of the material.

### DETAILED DESCRIPTION

One of the most challenging aspects of restoring and/or improving a native tissue's physiological function with engineered constructs is timing simultaneous transformation: the progression from synthetic to native structure. Though structural support for damaged tissue is essential^{[1, 2}], mechanical integrity can impact the functionality of host tissue (i.e. both soft and hard tissue).^{[3]} This is especially true for constructs that are not cellularized before implantation. Without native tissue ingrowth onto the implanted scaffold, specifically within the context of cardiovascular applications, physiological mechanical stresses can affect the durability of the scaffolds through repetitive flexion and extension cycles. This scaffold fatigue could be mitigated by introducing living cells into the scaffold's structure that are then capable of ECM repair and remodeling.

When designing functional scaffolds, fundamental requirements must first be considered in order to achieve a durable, non-thrombogenic tissue with growth potential. The scaffold must: 1) imitate native mechanical (elasticity and deformation) and structural properties (extracellular matrix (ECM) fiber alignment)^{[4-6]}, 2) facilitate cellular growth, tissue formation and vascularization^{[1, 7]}, and 3) possess controlled biodegradability^{[6, 8]}. Previous attempts to design synthetic scaffolds from polymers have captured a number of these characteristics^{[5, 6, 9-14]}. However, many of these materials have other notable shortcomings including: inelasticity (e.g. polyglycolic acid and polylactic acid, PGA and PLA, respectively)^{[11]}, plastic deformation and slow degradation over time (e.g. polycaprolactone (PCL))^{[6]}, low porosity and resulting poor cellular penetration (Polyurethane (PU) sheets^{[13]}), and a lack of fibrous structure (Poly Glycolic sebasic acid (PGS)^{[[9, 10]}) or lack of anisotropic characteristics (e.g. poly-carbonate-/ester-urethane urea) (PCUU/PEUU)^{[14]} and Poly(3-hydroxybutyrate-co-4-hydroxybutyrate) (P(3HB-co-4HB))^{[13]}. In addition to synthetic materials, natural hydrogels, including collagen and fibrin hydrogels, are notable for their ease of fabrication and their superior cellular retention^{[15]} (due to the presence of natural protein, collagen fibers, and glycosaminoglycans^{[16, 17]}), yet they lack mechanical integrity and have proven to be difficult to suture.

Using newer fabrication techniques, fibrous scaffolds have shown improved mechanical properties and fiber alignment providing anisotropy similar to native tissue^{[6]}. Although, these techniques result in nano- and micro-fibers, they have demonstrated reduced porosity and have inhibited cellular penetration into the construct, preventing 3-Dimensional (3D) tissue formation^{[8, 12, 18]}. Therefore, integrating cells within the 3D structure of scaffolds remains a primary challenge. Cellular encapsulation within hydrogels has shown preliminary success in generating a cellularized 3D construct^{[19]}. The application of hydrogels for soft tissue regeneration has been reported extensively, particularly in the design and fabrication of cell laden materials for wound healing, implantable tissues and tissue repair^{[20]}. To control the hydrogel structure and mechanical properties, researchers have incorporated photodegradable moieties into the synthetic hydrogels. We recently tested multiple hydrogels and demonstrated inter alia that methacrylated gelatin (GelMa) hydrogel provided promising results for generating tissues and vascular networks within the hydrogel, with properties that could be modulated and optimized on the basis of timing the photopolymerization and cross-linking of GelMa^{[16, 21]}. Similar to many naturally based hydrogels, thrombogenicity, suboptimal mechanical properties, poor durability, and decreased cellular spreading, however, were limitations that also accompanied these acellular hydrogel materials.^{[22]} similar to other hydrogel based materials used for scaffolding for tissue engineering.

Various native tissues are comprised of dense ECM fibers as well as hydrogel like content. For example, native aortic and pulmonary valve leaflets are comprised of two dense ECM fibrous layers (of collagen and elastin proteins) and a hydrogel like layer (containing glycosaminoglycan protein). In this study, we attempted to integrate the advantages of both synthetic biocompatible polymers in the form of fibrous scaffolds (structure and mechanics) and hydrogels (cellular retention properties) to create a novel, hybrid scaffold applicable for various soft tissue engineering. We fabricated a microfibrous scaffold based on newly synthesized poly-4-hydroxybutyrate (P4HB)^{[23]}, with favorable biomechanical properties (for example, elasticity and deformation in the physiological range, e.g., 15-20% strain for native tissues), anisotropy, and more rapid degradation. We then addressed the issue of cellular ingrowth by integrating mesenchymal stem cells (MSCs) into the 3D fibrous structure of the scaffold using the photo-crosslinkable hydrogel, GelMa. The synergistic properties of P4HB and GelMa were combined to create a biomimetic hybrid scaffold (P4HB/GelMa) with desired biomechanics and a hospitable environment in which cells can grow and proliferate. Understanding the role of mechanical forces on cell behavior is critical for tissue engineering, so bioreactor systems have been designed to mimic the physiological and tissue-specific in vivo environment.^{[24, 25]} Following hybridization, we conditioned the cellularized scaffold in a stretch-flex bioreactor to further promote cell growth while evaluating the scaffold's endurance under mechanical stimulation. We have further evaluated the P4HB/GelMa and P4HB composite scaffolds as a valve material in a bioreactor in which the cell-seeded material (EPCs and MSCs) was subjected to pressure, flexion, and shear forces similar to those in the mammalian pulmonary circulation. The combination of an anisotropic, fibrous scaffold and a tunable, native-like hydrogel for cellular encapsulation enhanced the formation of 3D tissue and provided a biologically functional, hybrid scaffold for in vivo implantation.

Thus, provided herein are methods and compositions for use in making artificial tissues, that use a P4HB scaffold, preferably with a hydrogel such as photo-crosslinkable GelMa. These hybrid scaffolds can be seeded with stem cells, e.g., mesenchymal stem cells (MSCs), and optionally coated with endothelial progenitor cells (EPCs). In some embodiments, the cells are autologous to (derived from) a subject who is in need of a transplant; the cells can be induced pluripotent stem cells (iPSCs). The cell-seeded scaffolds are maintained under conditions such as those described herien to allow the cells to proliferate and form tissue. These artificial tissues, which can be shaped by altering the shape of the P4HB scaffold, can be implanted into a subject using known transplant methods, e.g., in place of a heart valve leaflet, vascular conduit or blood vessel, or a portion thereof, e.g., to treat subjects in need thereof. Subjects in need thereof can include, for example, subjects who have congenital cardiac or vascular malformations, or who have suffered trauma (either accidental or intentional, e.g., surgical) to a blood vessel or heart valve, or who are in need of artificial skin.

### EXAMPLES

The invention is further described in the following examples, which do not limit the scope of the invention described in the claims.

Methods

Experimental Section

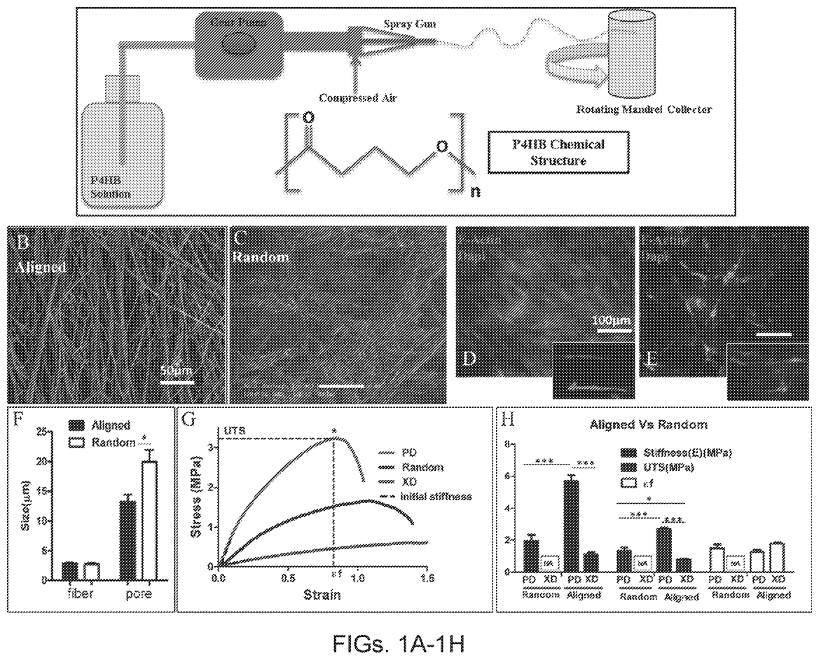

Poly-4-hydroxybutyrate (P4HB) (Mw=390 kDa, Tepha, Inc. Lexington, Mass.) was biosynthesized using a recombinant strain of Escherichia coli K12 and was isolated and purified as previously described.^{[23]} The chemical structure of P4HB is shown in FIG. 1A. Highly porous nonwoven scaffolds of P4HB were prepared with a novel dry spinning technique. In brief, P4HB was dissolved in chloroform (8% wt/vol) to create a viscous solution that was sprayed through an automatic spraying gun (Model RA 5, Krautzberger GmbH, Germany) using compressed air to draw and attenuate the fibers as they departed the spray nozzle. The solvent evaporates during the flight of the polymer strands to create continuous micron-sized fibers of consistent diameter (˜1.8 μm). The fibers were collected on a flat fiberglass filter at a working distance of 33″ from the spray nozzle to obtain the random nonwoven scaffolds. P4HB nonwoven scaffolds with highly aligned fibers and anisotropic properties were prepared by using a rotating mandrel collector (OD: 3.25″, working distance: 27″) with a rotational speed of 1166 rpm as shown in FIG. 1A.

GelMa was synthesized as described previously from type-A porcine skin gelatin (Sigma-Aldrich).^{[14]} The methacrylation process, under stirring conditions, is described in detail in the supporting information. The GelMa solution was dialyzed against deionized water, stored frozen at −80° C., lyophilized, and again stored in the freezer. Before use for cell seeding processes, a GelMa pre-polymer solution was prepared by dissolving the freeze-dried GelMa (5 w/v % final) and the photo initiator (Irgacure 2959) (0.5 w/v %, CIBA Chemicals) in DPBS at 60° C. Photocrosslinking was achieved by exposing the GelMa pre-polymer to 6.7 mW/cm² UV light (360-480 nm; using an OmniCure 52000 UV lamp (Lumen Dynamics)) for 20 s at room temperature.

The scaffolds were tested with a uniaxial mechanical tester (Instron 5542) to assess the mechanical characteristics of the unseeded scaffolds initially and after a 4-week culture period (soaked in medium). The samples were then sterilely prepared for cell seeding and soaked in media for 2 days. The detailed MSC and EPC isolation has been described in supporting information. Bone marrow samples were obtained from sheep femurs in ARCH (Animal Research Children's Hospital Boston). For EPC isolation blood was derived from sheep donor. The blood was aspirated into a heparinized syringe (20-40 ml blood drawn from the right femoral vein using 19-guage needle). The MSCs were seeded directly on the scaffolds or were suspended (1×10⁶/cm² of the scaffold in 80 μl) in the GelMa solution (500 mg GelMa, 10 ml PBS with 5% photo initiator dissolved in the PBS). Photocrosslinking was achieved by exposing the cell-pre-polymer mixture to UV light (360-480 nm) for 15 seconds. Thereafter, cell-laden hydrogels encapsulated in fibrous scaffolds were cultured in Dulbecco's Modified Eagle Medium (DMEM) for a week in static culture. Following al-week static seeding, 8 scaffolds (prepared as described in supporting information) were placed in the bioreactor for further culturing in flexure and stretch condition. For comparison, 8 more samples were retained for continued study in the static conditions. At the end of the culture time, samples were cut and prepared for the biochemical assays, including collagen and DNA assays, to assess the tissue formation and cellular proliferation. Samples were also fixed and cut for histology and immunohistochemistry.

GelMa Synthesis:

Briefly, type-A porcine skin gelatin (Sigma-Aldrich) was dissolved in Dulbecco's phosphate buffered saline (DPBS) (GIBCO) at 60° C. to make a uniform gelatin solution (10% (w/w)). Methacrylic anhydride (MA) (Sigma-Aldrich) was added to the gelatin solution at a rate of 0.5 mL/min under stirring conditions. Final concentrations of MA of 1, 5 and 10% (v/v) were used (referred to herein as 1M, 5M, and 10M GelMa). The mixture was allowed to react for 3 h at 50° C. After a 5-times dilution with additional warm DPBS, the GelMa solution was dialyzed against deionized water using 12-14 kDa cut-off dialysis tubes (Spectrum Laboratories) for 7 d at 50° C. to remove unreacted MA and additional by-products. The dialyzed GelMa solutions were frozen at −80° C., lyophilized, and stored at room temperature.

MSCs Isolation and EPCs Isolation:

Bone marrow samples were obtained from sheep femurs in ARCH (Protocol No. 13-10-2531R). Prior to the isolation process, the samples were preserved in isolation buffer (ACD solution and heparin sulfate (American Pharmaceutical Partners)) on ice. 15 ml of Ficoll-Paque Plus (Amersham Pharmacia) was added to each 50 ml Accuspin tube (Sigma-Aldrich, A2055) and spun for 1 min (1200 rpm) to sediment the Ficoll-Paque. The mononuclear cell layer was collected with a syringe and transferred into 50 ml conical tubes on ice. Every 10 ml of collected cells were mixed with 5 ml isolation buffer. The cell pellet was obtained following two sequential spinning and resuspension cycles in isolation buffer. The cells were then ready for cultivation and further harvest.

For EPC isolation, blood was derived from sheep donor. Blood was aspirated into a heparinized syringe (20-40 ml blood drawn from the right femoral vein using a 19-guage needle). The blood was collected in a 50 ml tube including 10 ml isolation buffer (9.9 g Sodium Citrate in 640 ml DI water, 3.6 g Citric Acid, 11.02 g Dextrose [D-(+)-Glucose], 750 ml water; filtered). 15 ml Ficoll-Paque plus (GE Healthcare Life Sciences, Product Code: 17-1440-02) was added to 50 ml Accuspin tubes and then spun at 1200 rpm for 1 min to sediment the Ficoll-Paque below the filter. 30 ml of blood/isolation buffer was then added on top of each Accuspin tube and spun at 2700 rpm for 15 min at room temperature. Following the centrifuging, the cell layer was collected with a pipette and transferred to a new 50 ml tube. We again added 5 ml of isolation buffer to every 10 ml of collected cell layer. The samples ware then spun at 2700 rpm for 5 min. Following removal of the supernatant, the cell pellets were resuspended in 10 ml isolation buffer and spun at 1200 rpm for 10 min. The pellets were resuspended again in 2 ml isolation buffer and 6 ml ammonium chloride (Sigma Aldrich, Catalog Number: 09685) was added to the suspension to lyse erythrocytes. The solutions were then incubated on ice for 5-10 min. 5 ml Isolation buffer was added in the last step and the solution was centrifuged for 5 min in 1200 rpm. Of note, if pellet still had a red color, the previous steps were repeated until all color has been removed. The mononuclear cell solutions were plated in 100 mm tissue culture in Hu Plasma Fibronectin (Milipore Sigma, FC010) coated plates and then placed in an incubator (37° C.). 2 hr after the plating, the unbound cell fractions were aspirated and the bound cell fractions were cultured in EBM-2 medium (Lonza, product code 190860) supplemented with the EGM-2 bulletkit (Lonza, CC-3162).

### Cell Seeding & Encapsulation of MSCs in GelMa Hydrogels:

In preparation for cell seeding, P4HB scaffolds were first sterilized by soaking in 70% ethanol for 30 min, followed by high intensity UV exposure (800 mW) for 3 min. The scaffolds were then soaked in culture medium prior to the cell encapsulation. The MSCs were suspended in the GelMa solution (500 mg GelMa, 10 ml PBS with 5% photo initiator dissolved in the PBS). MSCs were suspended at 1×10⁶/cm² within the scaffold in 80 μl of the GelMa solution. The solution was added on top of the scaffolds as shown in the schematic. Photocrosslinking was achieved by exposing the cell-pre-polymer mixture to UV light (360-480 nm) for 15 seconds. Thereafter, cell-laden hydrogels encapsulated in fibrous scaffolds were cultured in DMEM for a week in static culture. The scaffold samples for bioreactor were been placed between rubber bands prior to sterilization and then soaked in GelMa and exposed to UV light. Following 1 week static seeding, 8 scaffolds were placed in the bioreactor for further culturing in a flexure and stretch condition. For comparison, 8 more samples were kept for further study in the static condition.

Mechanical Testing:

Scaffolds were tested by uniaxial mechanical Instron machine (Model 5542, Norwood, Mass.) to characterize the scaffolds' and tissues' mechanical properties. Samples were cut into 15 mm by 5 mm rectangular strips. Geometric data was imported into the Blue Hill mechanical testing software and samples were stretched to failure using a 10 N load cell to measure the reaction force. The samples were loaded at a 7 mm/min extension rate. In addition, the tri-layer scaffolds were tested by biaxial mechanical tester (CellScale, BioTester) to characterize the scaffold's mechanical properties in PD and XD direction. Scaffolds were cut in 5 mm squares and tested in PBS at 37° C. The samples were stretched to failure using a 5 N load a 10 mm/sec extension rate.

We measured the initial modulus (0-15% strain region; equivalent to the Young's modulus for a linear elastic material for scaffolds). The ultimate tensile strength (UTS) and the strain-to-failure for the scaffolds were also measured.

Pore Size and Fiber Size Measurements:

The fiber sizes and pore sizes of the fibrous scaffolds was measured using the image J software. Using the line measurement tool, we were able to draw a line across the diameter of fibers and measure range of fibers in several images obtained from the scaffolds. For pore sizes, we used the tool to measure the pores diameter via drawing a circle around the area and measure the diameter with the software. An average of the range of these measurements was reported as pore sizes.

DNA, Collagen and GAG Assays:

Samples (˜2.5 by 2.5 mm) were cut from the cell-seeded scaffolds and weighed prior to the extraction of the ECM. The Sircol™ collagen assay kit (Biocolor LTd., United Kingdom) was used as per the manufacturer's protocol to quantify the collagen content that was synthesized following the 2- and 4-week cultivations. In order to extract the collagen, samples were placed in PCR tubes in 100 μL of extraction solution (0.5 M acetic acid and 1 mg/ml pepsin A in water) overnight on an orbital rocker at room temperature. GAGs were extracted utilizing the Sircol™ GAG assay kit (Biocolor LTd., United Kingdom). Briefly, the samples were soaked in a 1 ml solution of 4 M guanidine-HCl and 0.5 M sodium acetate overnight at 2-8° C. Following the extraction steps, ECM proteins (collagen and GAG content) were measured according to the protocol provided with the Sircol™ assay kits using a Genesys 20 spectrophotometer (Thermo Spectronic, Rochester, N.Y.).

DNA content was quantified on fibrous, microfabricated and tri-layered scaffolds at each specific time point by using a PicoGreen dsDNA quantification kit (Invitrogen) per manufacturer's instructions using a Spectramax Gemini XS plate reader (Molecular Devices, Inc., Sunnyvale, Calif.)[23,31]. Samples (˜2 mm by 2 mm) were first cut from the cell-seeded scaffolds and weighed. The samples were then incubated in microcentrifuge tubes with 1 ml of buffered 0.125 mg/ml papain solution (DNA extraction solution) for 16 hr in a 60° C. water bath before performing the PicoGreen assay.

Histology and Immunostaining:

Samples were first fixed in 4% PFA for 30 min, then rinsed in PBS, after which they were stored in 30% sucrose solution at 4° C. overnight. Then samples were rinsed with PBS and embedded in OCT (Finetek). Cryosections of 10 μm were cut and stored at −20° C. Sections were thawed for 30 min before performing hematoxylin and eosin (H&E) staining for general morphology. To visualize myofibroblast-like differentiation, cell-seeded scaffold sections were stained for alpha smooth muscle actin (a-SMA, mouse monoclonal 1A4, Dako) using immunofluorescence. Normal horse serum (4%) was used as blocking solution. AlexaFluor 488 labeled secondary goat-anti mouse (Invitrogen) served as the secondary antibody. Sections were coverslipped with DAPI-containing Vectashield mounting media to counterstain the nuclei. Images were taken with a Nikon iEclipse microscope equipped with a digital camera (Nikon Instruments, Melville, N.Y.).

The cell-seeded scaffolds were prepared for nuclei and F-actin visualization. Samples were first rinsed in HBSS and then fixed in 10% neutral buffered formalin (Sigma) for 20 min. The samples were then allowed to incubate at room temperature for 2 hr in 0.2% (v/v) Triton X-100 (Sigma) in Hank's Balanced Salt Solution (HBSS). The samples were then rinsed 3 times for 5 min each in 0.05% (v/v) Triton X-100 in HBSS and then blocked in 1% (w/v) bovine serum albumin (Sigma) and 0.05% (v/v) Triton X-100 in HBSS for 2 hr. Once the blocking was complete, samples were incubated for 3 hr in Alexa Fluor 488-phalloidin (1:40 (v/v) dilution of stock solution in 1% (w/v) bovine serum albumin and 0.05% (v/v) Triton X-100 in HBSS); Invitrogen). The scaffolds were then rinsed 5 times for 5 min each in HBBS and stored in the refrigerator overnight. The samples were then placed on glass slides and coverslipped with a drop of Vectashield mounting media with DAPI (Vector Laboratories, Inc., Burlingame, Calif.) to counterstain cell nuclei.

Thrombogenicity Assay:

Human platelet rich plasma concentrates with approximately 1,000,000 platelets/ml were obtained from ZenBio. Inc. NC. The platelets were spun down in 50 ml tubes (2700 rpm for 5 min). The pellet was resuspended in 500 μl of media which led to a concentration of roughly 100,000,000 platelets/ml. Scaffolds were washed with PBS and placed in 12 well plates. Samples were submerged in 400 μl of the platelet solution for 1 hr on a rocker in an incubator. Following the soaking process, samples were washed with PBS, fixed in 10% formalin for 20 min and immunohistology was conducted as described above using anti-human CD41 (Invitrogen Carlsbad, Calif.) (1:200 for 1 hr at 37° C.) as a primary antibody and anti-mouse Alexa568 (1:40 for 1 hr at room temperature) as a secondary antibody. Samples were stained with mouse anti-human CD41 (Invitrogen, Carlsbad, Calif.) (1:200 for 1 hr at room temperature). The samples were then washed and soaked in a solution of Alexaflour 568 anti-mouse (1:40 for 1 hr at room temperature).

Scanning Electron Microscopy (SEM) and Confocal Microscopy:

Scaffolds were imaged at different magnifications (e.g., 50×, 100×) using an environmental scanning electron microscope (ESEM), SEMXL30 at low vacuum with a 32 kV accelerating voltage, 11 mm working distance. Immunohistology was visualized using a fluorescence microscope equipped with florescence camera (Axio Cam. MRm) and manufactured ApoTome for depth imaging (Carl Zeiss MicroImaging, Gottingen, Germany).

Surgical Implantation

The animal (Dorsett sheep) was pre-medicated with atropine 0.04 mg/kg IM followed by ketamine 10 mg/kg and versed 0.1 mg/kg IV. Following this, the animal was intubated with and endotracheal tube, and general Isoflurane anesthesia was administered. A 10 French Foley bladder catheter was inserted directly into the urethra and a 6 French percutaneous arterial catheter was placed in the right femoral artery for arterial pressure monitoring. A 7 French triple lumen venous catheter was inserted in the right external jugular vein. To control ventilation and allow hemostatic transection of the muscular layers of the chest, cisatracurium was administered to achieve reversible muscular paralysis. Heart rate and blood pressure were monitored to ensure deep anesthesia while the animal was paralyzed. The animal was continuously monitored by the following parameters: arterial blood pressure, central venous pressure, heart rate and rhythm, oxygenation, temperature and urine output. Ancef 20 mg/kg IV was additionally given for antimicrobial prophylaxis.

The left thorax was prepared by shearing and painting with Betadine, and was draped using sterile drapes, and an anterolateral left-sided thoracotomy was performed in the 3rd intercostal space. With the lung retracted posteroinferiorly, the pericardium was opened longitudinally to expose the main pulmonary artery. A segment of main pulmonary artery was isolated with a partial occlusion clamp above the sinotubular junction. The pulmonary artery was then incised longitudinally (2 cm) and the patch material (P4HB/GelMa) 2 cm×1.5 cm was sutured into the incision site as an only patch. After hemostasis was ensured, the partial occlusion clamp was removed, chest tubes were placed (one in the left pleural space and the other behind the base of the heart), and secured to the skin. Intercostal sutures (0-vicryl) were placed to approximate the ribs. An intercostal block was placed using 0.25% sensorcaine 1 mg/kg. Soft tissue and skin were closed using PDS (4-0/2-0) and monocryl 4-0, respectively. Dermabond was administered over the wound. Subsequently, the sheep recovered form anesthesia and was returned to housing.

Experimental Section

Poly-4-hydroxybutyrate (P4HB) (Mw=390 kDa, Tepha, Inc. Lexington, Mass.) was biosynthesized using a recombinant strain of Escherichia coli K12 and was isolated and purified as previously described.^{[23]} The chemical structure of P4HB is shown in FIG. 1A. Highly porous nonwoven scaffolds of P4HB were prepared with a novel dry spinning technique. In brief, P4HB was dissolved in chloroform (8% wt/vol) to create a viscous solution. For the tri-layered valve, P4HB was dissolved in Hexafluoro-2-propanol (HFIP, 8% wt/vol) for the outer layers and, P4HB-Gelatin (porcine skin type A) was dissolved in a 1:1 ratio ((12% wt/vol) in HFIP. The solutions were sprayed through an automatic spraying gun (Model RA 5, Krautzberger GmbH, Germany) using compressed air to draw and attenuate the fibers as they departed the spray nozzle. The HFIP solvent evaporated during the flight of the polymer strands to create continuous micron-sized fibers of consistent diameter (˜1.8 μm). The fibers were collected on a flat fiberglass filter at a working distance of 33″ from the spray nozzle to obtain the random nonwoven scaffolds. P4HB nonwoven scaffolds with highly aligned fibers and anisotropic properties were prepared by using a rotating mandrel collector (OD: 3.25″, working distance: 27″) with a rotational speed of 1166 rpm as shown in FIG. 1A.

GelMa was synthesized as described previously from type-A porcine skin gelatin (Sigma-Aldrich).^{[14]} The methacrylation process, under stirring conditions, is described in detail in the supporting information. The GelMa solution was dialyzed against deionized water, stored frozen at −80° C., lyophilized, and again stored in the freezer. Before use for cell seeding processes, a GelMa pre-polymer solution was prepared by dissolving the freeze-dried GelMa (5 w/v % final) and the photo initiator (Irgacure 2959) (0.5 w/v %, CIBA Chemicals) in DPBS at 60° C. Photocrosslinking was achieved by exposing the GelMa pre-polymer to 6.7 mW/cm² UV light (360-480 nm; using an OmniCure 52000 UV lamp (Lumen Dynamics)) for 20 s at room temperature.

The scaffolds were tested with a uniaxial mechanical tester (Instron 5542) to assess the mechanical characteristics of the unseeded scaffolds initially and after a 4-week culture period (soaked in medium). The samples were then sterilely prepared for cell seeding and soaked in media for 2 days. The detailed MSC and EPC isolation has been described in supporting information. Bone marrow samples were obtained from sheep femurs in ARCH (Animal Research Children's Hospital Boston). For EPC isolation blood was derived from sheep donor. The blood was aspirated into a heparinized syringe (20-40 ml blood drawn from the right femoral vein using 19-guage needle). The MSCs were seeded directly on the scaffolds or were suspended (1×10⁶/cm² of the scaffold in 80 μl) in the GelMa solution (500 mg GelMa, 10 ml PBS with 5% photo initiator dissolved in the PBS). Photocrosslinking was achieved by exposing the cell-pre-polymer mixture to UV light (360-480 nm) for 15 seconds. Thereafter, cell-laden hydrogels encapsulated in fibrous scaffolds were cultured in Dulbecco's Modified Eagle Medium (DMEM) for a week in static culture. Following al-week static seeding, 8 scaffolds (prepared as described in supporting information) were placed in the bioreactor for further culturing in flexure and stretch condition. For comparison, 8 more samples were retained for continued study in the static conditions. At the end of the culture time, samples were cut and prepared for the biochemical assays, including collagen and DNA assays, to assess the tissue formation and cellular proliferation. Samples were also fixed and cut for histology and immunohistochemistry.

### Example 1. P4HB Demonstrates Favorable Mechanics and Structural Anisotropy

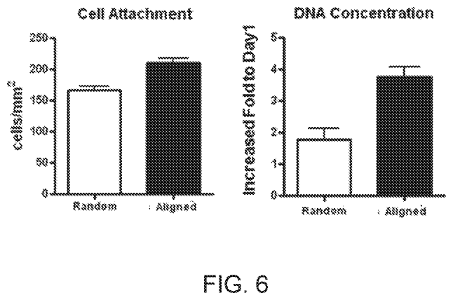

Fiber alignment was created using a rotating mandrel as a collector during a dry-spinning procedure at a speed of 1,166 rpm (FIG. 1A). Fibers were better oriented in the aligned scaffolds versus random scaffolds as shown in Scanning Electron Microscope (SEM) images (FIGS. 1B-C). This resulted from the variations in the collector of the dry spinning procedure (rotating mandrel vs. stationary flat collector). Random fibers were generated by dry spinning the raw material onto an immobile conductive surface, which pulled fibers in various directions. Aligned fibers were generated by spinning raw material onto a rotary mandrel rotating, perpendicular to the angle at which raw material was ejected from the source needle. Fiber alignment for both random and aligned fibers was quantified using ImageJ graphical analysis of SEM images. Aligned fibers showed increased cellular alignment of the MSCs, indicated by the F-Actin stain (FIGS. 1D-E). Pore and fiber sizes of scaffolds were also measured via SEM (FIG. 1F). While the fiber diameters remained similar in both scaffolds, larger pore sizes were observed in the random scaffolds versus aligned scaffolds (19.92±7.8 vs. 13.2±6.5 μm). This is most likely the result of random fiber orientation leaving unequal distributions between fibers. With average cell size ranging from 10-30 μm^{[10]}, our fibers provided a surface large enough for cell attachment and alignment, while pore size was concomitantly large enough to permit potential cell passage into the 3D structure of the scaffold (FIG. 1F). Fiber alignment enhanced cellular attachment and promoted growth and proliferation (FIG. 6).

A fundamental requirement for TE scaffolds is to provide a mechanically tolerant material capable of withstanding the physiological stress and strain of a relevant tissue^{[26]}. Mechanical properties of random and aligned P4HB were assessed with uniaxial testing. Stress-strain curves of random and aligned scaffolds were obtained, followed by measurement of the initial stiffness through the slope of the curves (at 15% strain), at the point of failure for the Ultimate Tensile Strength (UTS), and at strain-to-failure (εf) (break point denoted with *) (FIG. 1G-H). Stress-strain curves demonstrated different anisotropic properties between the preferred directions (PD)(aligned with fiber directions), and the cross-orthogonal direction (XD) for the aligned scaffold. This difference, however, was not observed for the randomly oriented scaffold fibers, which had similar mechanical properties in all directions (FIG. 1G-H). Aligned scaffolds possessed higher UTS (2.68±0.2 MPa) and lower εf (1.27±0.23) in the PD direction. These studies revealed that the scaffold was stiffer in the direction of fiber alignment (PD) (stiffness (E)=5.68±0.75 MPa) and more deformable in the XD direction (εf=1.8±0.13). Moreover, the aligned arrangement of fibers improved the scaffold's mechanical properties in terms of stiffness and UTS. Anisotropy is essential to scaffold characteristics, especially in cardiac tissues, as native tissues' ECM protein fibers are aligned in specific directions^{[8, 12, 27, 28]}. For example, the opening of heart valve leaflets during systolic blood flow and closure during diastole depends on the elasticity and anisotropy of this tissue^{[29]}. Myocardial stretching during the cardiac cycle also relies on tissue flexibility and anisotropy. Blood vessel elasticity modulates circulatory pressures and depends on tubular contraction and undulation, essential components that are dependent on structural anisotropy. Previous studies of other synthetic biomaterials have also shown the significance of scaffold architecture and fiber orientation in relation to biomechanics, cellular attachment and alignment.^{[6, 10, 12, 30]} Thus, the anisotropic characteristic of our aligned P4HB scaffold was reasonably similar to that of some native tissues.^{[28, 30]}

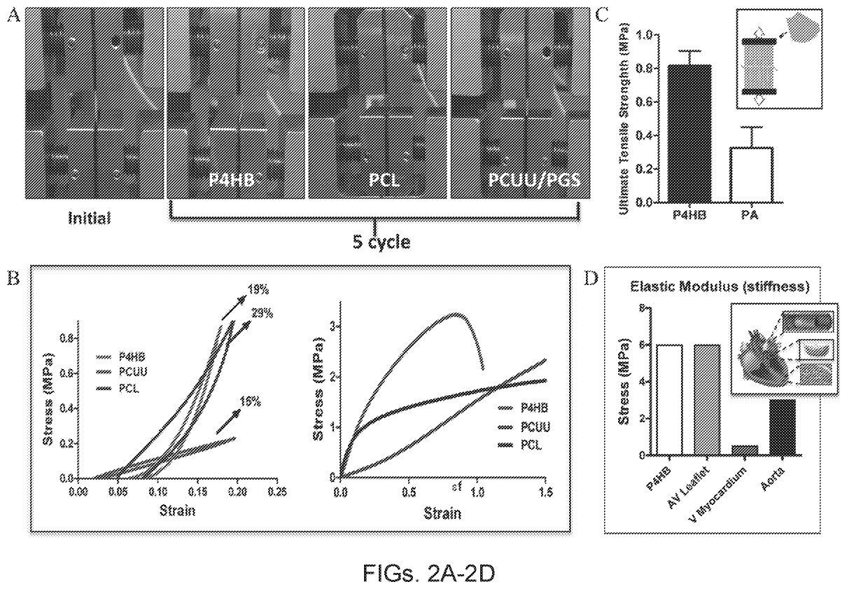

Cyclic tensile tests were performed up to a maximum of 20% strain—which corresponds to the range of physiological deformation^{[31]}—to evaluate the elasticity of P4HB in comparison to previous scaffolds^{[6, 27, 32]}. The initial position of the scaffolds (left panel), prior to starting the subsequent 5-cycle tensile tests (right 3 panels) for each of the materials, was compared (FIG. 2A). PCL scaffolds deformed considerably following the cycles due to its inherent plasticity. However, for relatively elastic materials, like PCUU, no deformation was observed following cyclic tests. Of note, no significant deformation was observed for P4HB, which suggested favorable elastic properties.

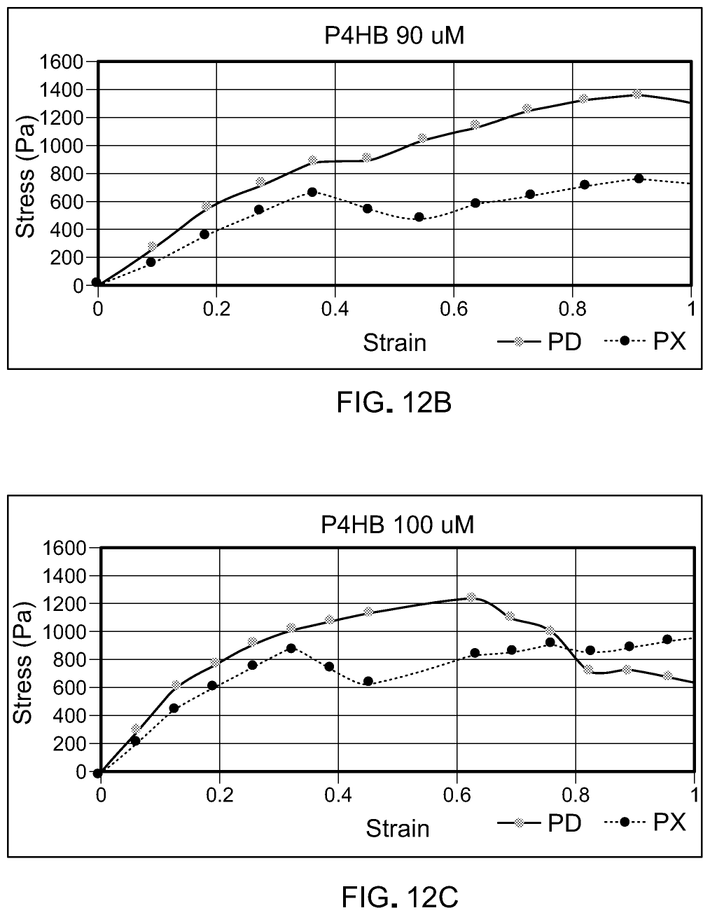

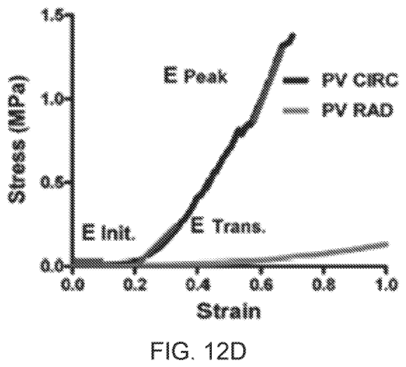

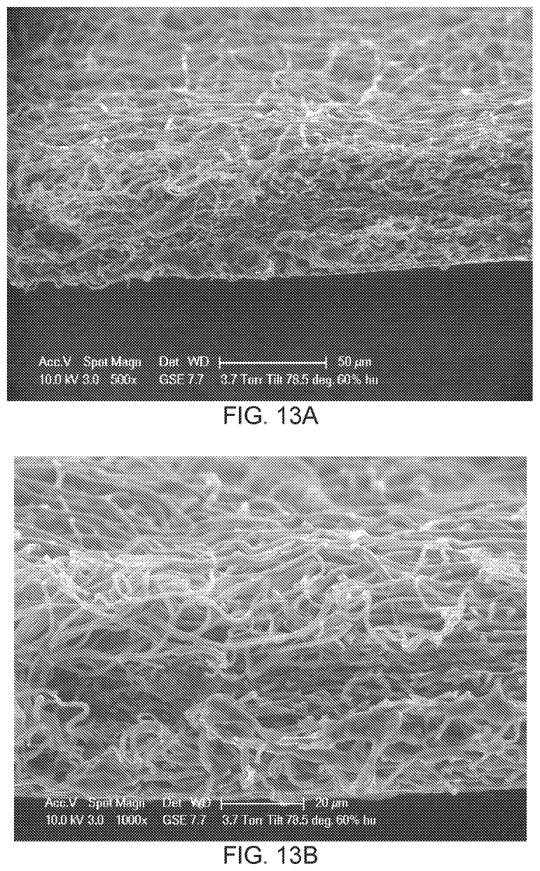

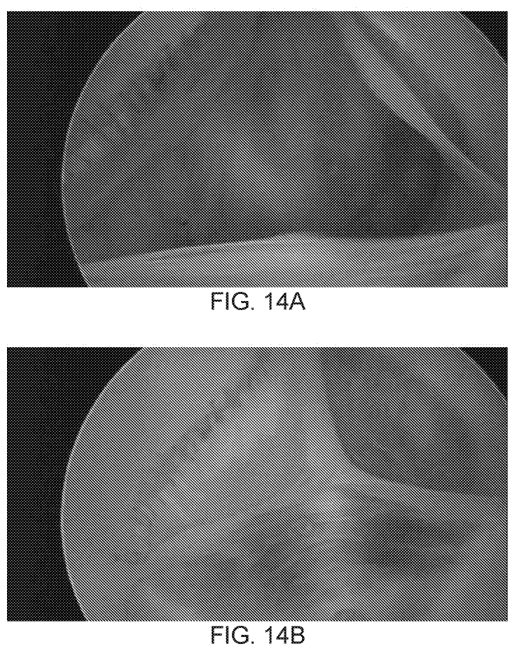

We next obtained stress-strain curves for the aforementioned cyclic tests (FIG. 2B). As expected, P4HB demonstrated low energy loss (19%), which was comparable to PCUU (15%), and higher resilience to deformation compared to PCL (29%). This finding is in accordance with the linear trend of the stress-to-strain curve of P4HB, which revealed no evidence of plastic deformation when compared to the PCL stress-to-strain curve. These results were notable considering that elasticity is a property that is indispensable to the functionality of many native tissues.^{[4, 28, 33]}By comparison, the energy loss of native aortic and pulmonary valve leaflets (defined as the area under the stress/strain curve, during the stretch and return of a cycle) (˜20%) is similar to that found in our P4HB scaffolds, suggesting comparable elasticity and deformability between our scaffolds and those native structures.^{[33]} In contrast, the P4HB bulk materials have relatively low elasticity^{[23]} and higher stiffness (reported as tensile modulus 70 MPa and UTS 60 MPa^{[23]}) when they were evaluated as sheets as opposed to the multiple fiber structure in our material. This difference indicated that fabrication of the P4HB as fibrous constructs resulted in more flexible and elastomeric materials. In addition, scaffolds ranging from 80 μm to 100 μm thickness were tested by a biaxial mechanical tester (cell scale) and showed similar stress-strain curves under 0.3 strain as native tissue. This indicates shows that variable thickness does not influence the strength of the scaffold (see FIGS. 12A-12B).

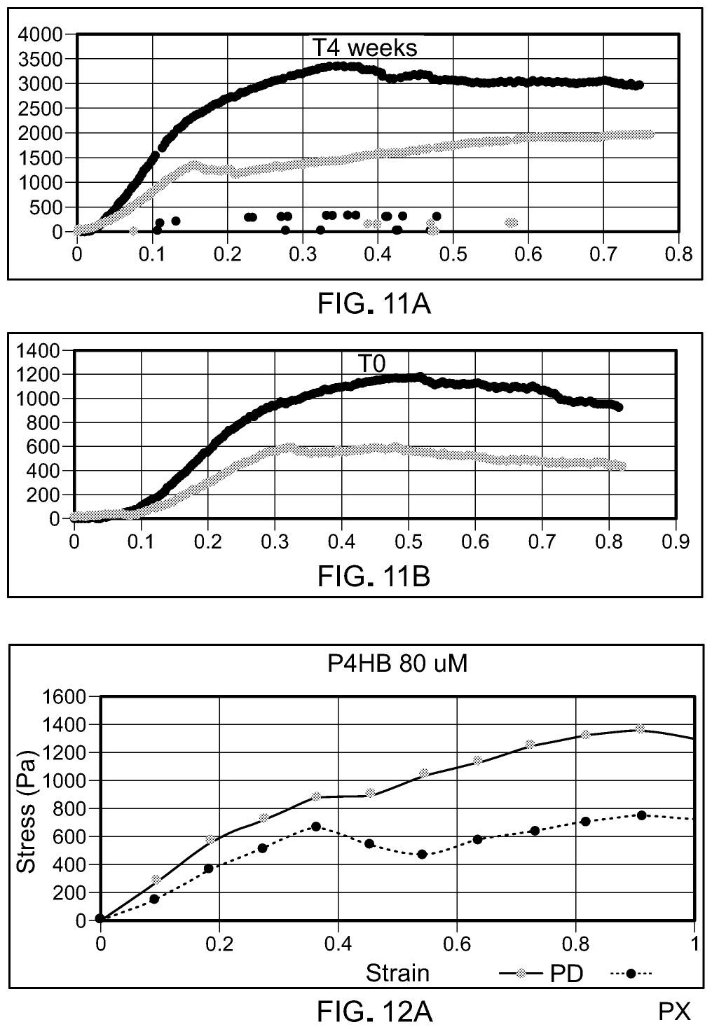

Further, seeding the P4HB composite scaffold with EPCs only on the outside showed the anisotropic properties were maintained after 4 weeks of static culture. While the thickness of the scaffold was decreased by 26% both UTS, in PD (1.17 to 3.32 MPa) and XD (0.59 to 1.34 MPa) and the stiffness E in PD (3.94 to 18.50 MPa) and XD (1.91 to 9.26 MPa) were increased, suggesting the cells made connections and/or produced ECM to strengthen the scaffold. (See FIGS. 11A-11B)

While providing favorable structure and mechanics, synthetic scaffolds, when compared to natural hydrogels, may not be preferable, either in terms of cellular attachment or tissue ingrowth.^{[5]} One of the concerns regarding fibrous scaffolds is variation in pore sizes (some too small and some too large)^{[18]}, which can impair cellular ingrowth within the 3D structure. If the pores are too small, cells cannot penetrate, but if the pores are too large, cells on adjacent fibers are sufficiently distant from one another to impair tissue formation. We hypothesized that filling the porous scaffold with a hydrogel, to create a hybrid structure, would overcome these problems of varying pore size on cell growth. In a sense, filling scaffolds with cellularized hydrogels “decouples” the need for a scaffold with defined mechanical properties from its ability to attract cells. We reasoned that introducing GelMa into the fibrous structure of P4HB would result in a hybrid P4HB/GelMa to provide not only a cell compatible environment but also one that would enhance cell growth throughout the 3D structure.

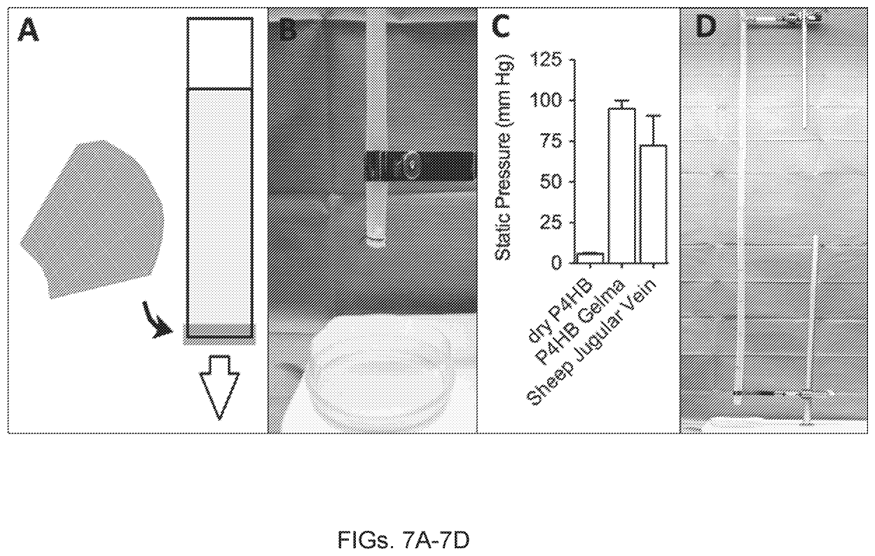

In addition to assessing this hybrid for its ability to incorporate cells, however, we tested P4HB/GelMa for its ability to hold suture and prevent fluid leakage under hydrostatic pressure. This mechanical property is a particularly important factor to consider in cardiovascular tissue engineering^{[33, 34]} Retention tests were performed and UTS values at the point of failure were measured for bare P4HB (0.81±0.15 MPa) and compared to that of the pulmonary artery (0.32±0.21 MPa). Scaffolds were capable of holding sutures while maintaining shape under physiological stress equivalents (FIG. 2C). Perfusion pressure tests indicated that scaffolds embedded with GelMa withstand hydrostatic pressures comparable to the results obtained from those of sheep pulmonary artery (FIGS. 7A-7D). These results were favorable compared to those of bare P4HB, which demonstrated a rapid leakage of fluid through the scaffold's fibrous structure. These mechanical properties for bare P4HB (E=6 MPa) were also compared to the stiffness of other cardiac tissues, including aortic valve leaflet (E=6 MPa)^{[30, 33]}, ventricular myocardium (E=0.5 MPa)^{[35]}, and aorta (E=3 MPa)^{[36]}. The elastic modulus of P4HB, on the order of 7 MPa, is comparable to that of the valve leaflet. These results suggest that it could also serve as a potential replacement for vascular conduits and blood vessels. (FIG. 2D)

### Example 2. P4HB/GelMa Scaffolds Encapsulate and Maintain Cell Viability

Protein-based hydrogels have been utilized for different regenerative medicine applications because of their amino acid composition and their potential for supporting biocompatibility in in vivo environment^{[37]}. To avoid the water solubility, these hydrogels require crosslinking reaction to stabilize the protein content within the hydrogel for in vitro or in vivo application.^{[16]} Prior investigators have proposed using physical or chemical crosslinking processes to overcome these challenges. However, physical crosslinking while capable of rapid gelation requires unique crosslinking conditions (due to sensitivity to temperature, PH or ionic concentration) that would limit the use of this method for in vivo applications.^{[38]} Chemical crosslinking, allows for the formation of permanent irreversible bonds between chemically active functional groups in the protein sequence^{[39]} (Producing crosslinks between native groups such as amines, carboxyls, and sulfhydryls with addition of a crosslinker, for instance, glutaraldehyde). However, controlling the physical properties to tune the degradation rate is limited due to long reaction times, preventing their applications in circumstances where rapid gelation or degradation is required. Also the toxic byproducts of the chemical crosslinking techniques have been reported to be problematic.^{[37]} Using the photocrosslinking method in this study, we were able to form the chemical bonds within seconds and tune the physical and chemical properties of GelMA hydrogel by varying the UV radiation parameters (e.g, time and energy). Moreover, photocrosslinking technique allows for spatial and temporal control of crosslinking that facilitates the hydrogel fabrication and application.

In the past,^{[21]} to fabricate GelMA with tunable mechanical characteristic, three different GelMA hydrogels were synthesized using 1M, 5M, and 10M methacrylic anhydrate. The actual percentages of the functionalized methacrylation groups were determined by measuring the extent of free amine group substitution using 1H-NMR spectroscopy. The degree of methacrylation (defined as the ratio of functionalized to original amino groups) corresponded to 49.8%, 63.8% and 73.2% for the 1M, 5M and 10M GelMA hydrogels, respectively and as expected, the compressive modulus of the GelMA increased with the degree of methacrylation. By measuring the percentage of hydrogel residual mass as a function of time, the degredation rate of the hydrogel was also determined. We found that the rate of degradation decreased with the methacrylation degree of the GelMA (1 M GelMA hydrogels were completely degraded within 6 h, whereas the 10M GelMA hydrogels lasted for 15 h). For this study, as explained further in this section, we used the 1M GelMA hydrogel to achieve a rapid degradation rate and using the hydrogel as cell carrier. In addition, we have shown that a liquid solution of GelMA injected beneath the skin can undergo polymerization rapidly (15-30 s) after this injection while continuing to support human progenitor cells and MSCs.^{[16]}

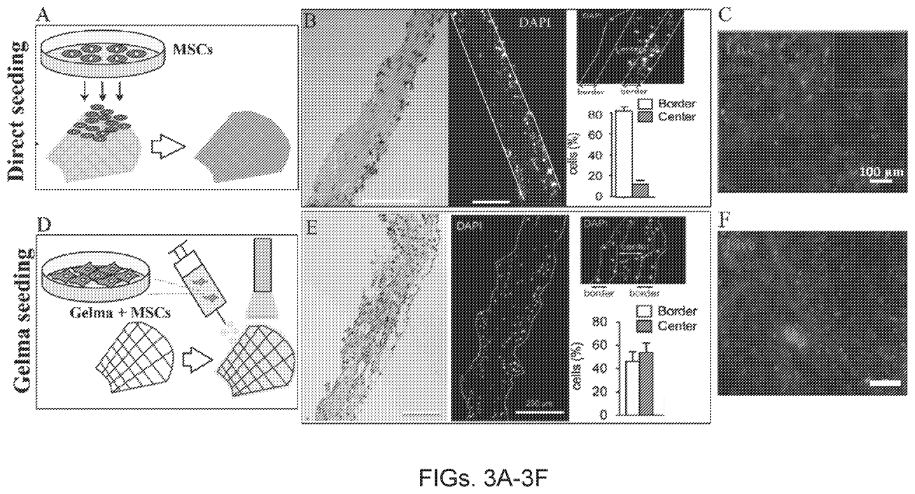

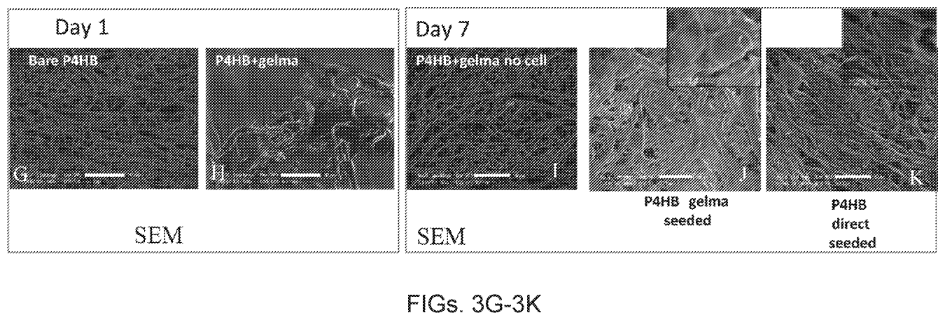

In our study, two processes of seeding were utilized, direct surface seeding and encapsulation of cells into GelMa prior to addition to the scaffold. These two methods resulted in varied patterns of subsequent tissue formation (FIGS. 3A-F). The first schematic in FIG. 3A shows a general 2D surface seeding of MSCs onto bare P4HB scaffolds. Seven days after seeding, histological evaluation of nuclei and quantitative analysis of cell infiltration revealed that surface seeding on bare scaffolds produced a cellularized surface but no significant cell penetration into the 3D construct or tissue growth (FIG. 3B). In contrast, as shown in the second schematic, MSCs that were encapsulated in GelMa prior to exposure to the scaffold penetrated the 3D structure of the P4HB scaffold. After 7 days in the P4HB/GelMa hybrid construct, cells not only grew along the surface of the scaffold but they also penetrated into the 3D structure of the scaffold (FIG. 3E). Both methods of seeding (2D and 3D) retained cell viability, as demonstrated by Live/Dead assays (FIGS. 3C and 3F, respectively).

Cellular encapsulation with hydrogels to create a 3D-tissue environment has previously been reported as a technique in tissue engineering^{[40]} However, for load bearing tissues, soft hydrogels alone do not satisfy the mechanical strength and anisotropic requirements of relevant tissues. We showed that successfully integrating cells into fibrous P4HB with GelMa resulted in a 3D cell seeding without significantly affecting the mechanical properties of the scaffold. Of note, the requirements of both synthetic materials and hydrogels were decoupled with our combination of P4HB/GelMa. On the one hand, fibrous scaffolds often present an environment that is difficult for cells to penetrate in a 3D manner; on the other hand hydrogels do not provide sufficient mechanical strength for certain tissue engineering applications. In this regard, P4HB/GelMa is a novel material that combines the properties of a mechanically favorable, fibrous scaffold with those of a hydrogel that can encapsulate cells for growth within a 3D environment.

### Example 3 Structural Microscopy Confirms Retention of Cells in P4HB/GelMa Scaffolds

We compared the structure of bare P4HB and P4HB/GelMa at 1-day of culture using SEM (FIGS. 3G-H). Scaffolds with GelMa showed a smoother surface structure as the gel permeated the scaffold pores and created a homogenous layer of GelMa on the surface and throughout the fibers (FIG. 3H). A series of experiments were performed to obtain the optimum GelMa stiffness to optimize spreading and attachment of cells during cultivation. Results confirmed that an increase in the degree of crosslinking of GelMa, obtained by longer UV exposure or higher UV intensity, impaired cell spreading. Similar results were reported in a recent study of encapsulated valvular interstitial cells in GelMa.^{[19]} Moreover, a large number of the cells did not remain attached to the scaffolds after mechanical stimulation, which could have resulted from the connection of the cells to the hydrogel instead of P4HB. To overcome this limitation and preserve cell and tissue formation throughout the scaffold, we optimized the degree of crosslinking for GelMa to provide the scaffolds with a specific stiffness. At our point of optimization, GelMa formed and encapsulated the cells within the scaffold's 3D structure, but the GelMa then degraded during a 1-week static culture. This degree of crosslinking also allowed cells to attach to P4HB scaffold's fibers and spread throughout the interior layers of the scaffold compared to penetration with increasingly solid GelMa structures.

Following 7 days of culture, the scaffolds from each of the seeded conditions were fixed for SEM imaging (FIG. 3I-K). When GelMa, without cells, was added to scaffolds, GelMa disappeared almost completely after 7 days and the underlying fiber structure was visualized (FIG. 3I). When P4HB was seeded with both GelMa and cells, tissue formation appeared and was distributed more evenly (compared to when P4HB was seeded with MSCs directly on the surface) as GelMa disappeared over the 7-day timeline (FIGS. 3J-K). Following 7 days, the similarity between the structure of the bare P4HB scaffolds and P4HB/GelMa without cells suggested that GelMa had degraded after 7 days of incubation (FIGS. 3G and I). Additionally, the proliferation of cells and concomitant disappearance of GelMa (in FIG. 3J) was confirmed when compared to the empty pores of P4HB/GelMa without cells (in FIG. 3I). These results suggested that GelMa played a role in enhancing cell penetration into the 3D structure of P4HB but degraded after 7 days and thus did not prevent cell growth and proliferation as shown by the DNA assay, quantifying cell number, explained in following section.

### Example 4 Cell-Seeded P4HB/GelMa Scaffolds Affect Mechanical Properties and Tissue Formation in In Vitro Bioreactor Conditions More than in Static Culture

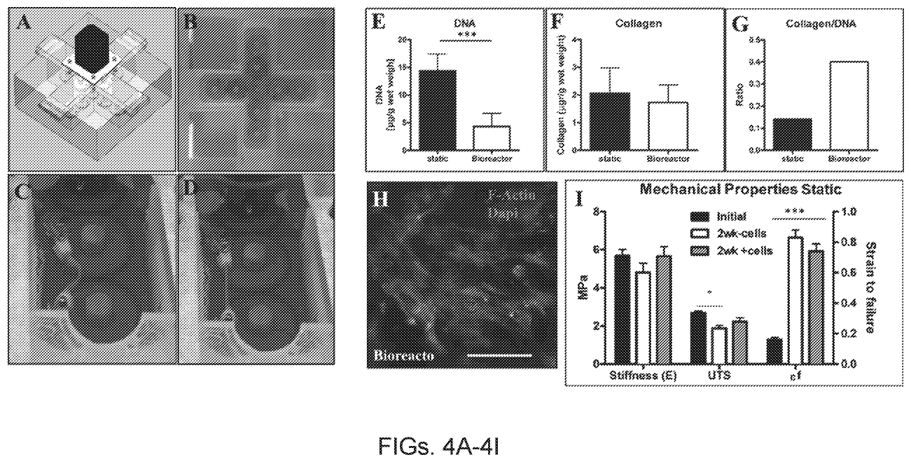

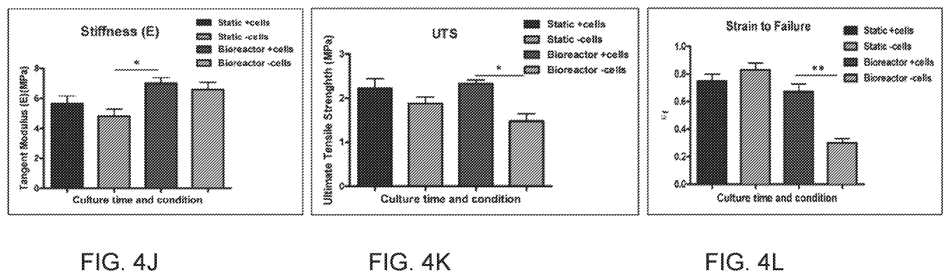

Following examination of mechanical properties and integration of cells throughout the 3D structure of the scaffolds, P4HB/GelMa was tested in a stretch/flex bioreactor that we previously designed and tested for growing fibroblas and valvular interestitial cells.^{[24]} The scaffold's deformation and tissue formation were then assessed after exposure to physiological stresses and flexure. Schematic, design, and functional portraits of the stretch-flex bioreactor used in this study are depicted, respectively, in FIGS. 4A-D. More specifically, FIGS. 4C-D show P4HB/GelMa scaffolds in the flex (C) and stretch configurations (D). The detail of the scaffolds' culture in the bioreactor can be found in the supporting information. Scaffolds were seeded and cultured statically (7 days) prior to implanting them in the bioreactor, for another 7 days. Samples were stretched and flexed initially with a lower strain rate (1 cycle/3.5 sec) compared to physiological rates (1 cycle/1.3 sec) to allow the cells to acclimate to the imposed mechanical stress environment. This accommodation protocol prevented loss of cells during the initial time of cultivation in the bioreactor, which had been seen in pilot experiments where samples were initially stretched and bent with a higher strain rate (data not shown).

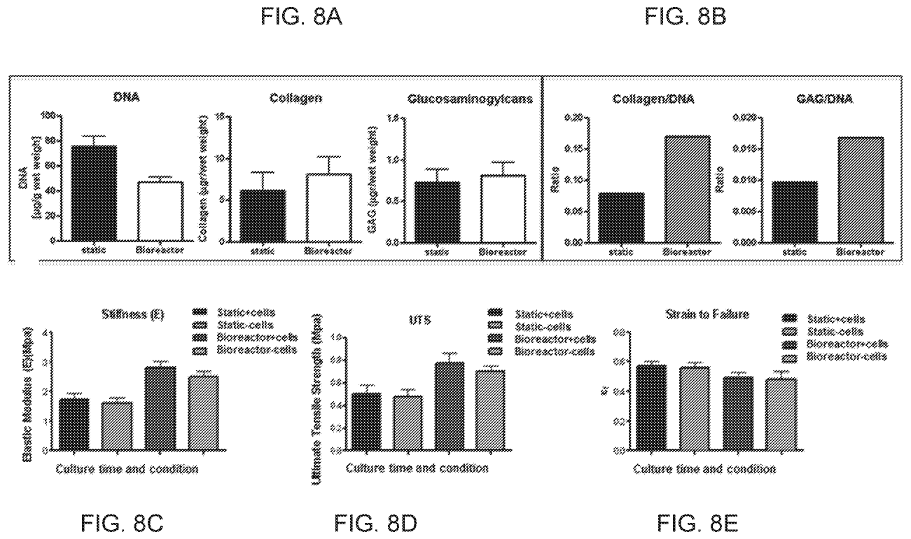

To evaluate the effect of 15% stretch and 20% flexure (based on radius of curvature as we previously described in the design of the bioreactor^{[19]}) on tissue formation on the scaffolds, samples were assessed with biochemical assays (i.e. DNA and collagen content) and compared to the statically cultured samples. Variations in DNA and collagen content between static scaffolds and those stretched and flexed in the bioreactor are denoted in FIGS. 4E-G. Results confirmed that the static condition produced more DNA (14.35±3.09 vs. 4.29±2.41 μg/gr wet weight), but the amount of collagen produced was not statistically different compared to that of the bioreactor condition (2.06±0.92 vs. 1.72±0.64 μg/gr wet weight). The average value of DNA obtained from the bioreactor condition was comparable to the DNA obtained from the samples that were cultured in a static condition for 7 days, prior to bioreactor implantation (average of 4.5 μg/gr wet weight). This finding indicates that DNA did not increase significantly in the bioreactor condition versus the static condition. Therefore, the lower amount of DNA in samples from the bioreactor, was likely due to the enzymatic activities of the cells that produced ECM production rather than cell proliferation. Also, the ratio of total micrograms of collagen produced per microgram of DNA, which was higher in the bioreactor samples than the static samples, supports the aforementioned conclusion (FIG. 4G), and is consistent with previous studies.^{[24, 41]} Thus, fewer cells, which correlate to the lower amount of DNA, produced a larger total mass of collagen rather than an increased number of cells (and DNA) being responsible for increased collagen levels. Similar results were obtained when random P4HB scaffolds were seeded with a higher density of MSCs. The to finding that cell seeding onto aligned fiber scaffolds resulted in increased stiffness and UTS (E=5.66±1.24 MPa and UTS=2.22±0.53 MPa) and deformation (εf=5.92±0.96) compared to unseeded aligned fiber scaffolds seems likely to be the result of tissue production in the cell-seeded scaffolds (FIG. 4). This finding was present in both static and dynamic culture environments. The trend was similar for the effect of cell seeding on random fiber scaffolds (FIGS. 8A-8E).

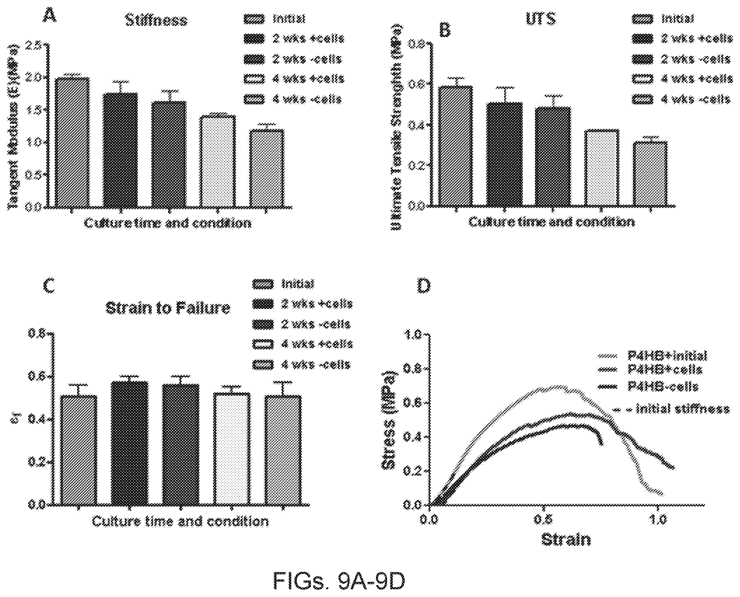

F-actin staining was used to evaluate the presence and adequate spreading of MSCs on P4HB after one week of static seeding, followed by 7-day cultivation in the stretch/flex bioreactor (FIG. 4H). Mechanical properties were assessed following 14-day static culture to evaluate the effect of tissue formation on the mechanical properties of the P4HB scaffolds. Data was compared with initial and control (non-seeded) mechanical properties (FIG. 4I). The initial stiffness of the cell-seeded P4HB-GelMA scaffolds (5.68±0.75 MPa) and UTS (2.68±0.2 MPa) was greater than uncellularized conditions following a 2-week culture (E=4.81±1.19 MPa and UTS=1.88±0.34 MPa), which indicated a degradation of the scaffolds during the culture period. However, the improved mechanical properties (E=5.66±1.24 MPa and UTS=2.22±0.53 MPa) and deformation (εf=5.92±0.96) of seeded scaffolds versus unseeded scaffolds and initial condition confirmed the presence of tissue formation and ECM production (see FIGS. 9A-9D for similar results of random scaffolds cultured in static conditions). We hypothesized that the reductions in stiffness and UTS that were observed in both seeded and unseeded random fiber scaffolds represent surface hydrolysis of the P4HB fibers. This finding was offset to some degree (not statistically significant) by the presence of cells on the random fibers as in FIGS. 9A-9D).

Biomechanical tests, for both seeded and unseeded conditions, were repeated for bioreactor samples and compared with static conditions (FIGS. 4J-L). The stiffness for unseeded samples increased in the bioreactor (6.58±1 MPa), suggesting an induced alignment of fibers as the fibers were stretched with a resulting change in overall stiffness (FIG. 4J). Similar results were found when random fibers were implanted in the bioreactor (FIGS. 8A-8E). Higher values of stiffness for seeded samples (6.99±0.87 MPa) in the bioreactor, versus static conditions, seemed to correspond with higher collagen/DNA values.

The ultimate tensile strength (UTS) for the bioreactor samples with cells remained unchanged when compared with the static scaffold data (2.33±0.19 MPa). The bioreactor samples without cells, in contrast, showed a slight decrease in UTS (1.48±0.33 MPa) when compared to the unseeded static samples (FIG. 4K). Analogous to the UTS data, bioreactor samples with cells showed similar deformation when compared with statically seeded scaffolds in strain-to-failure measurements (0.75±0.12 MPa vs. 0.67±0.14 MPa). Bioreactor samples without cells, however, showed decreased strain-to-failure properties compared to static samples, as well as seeded bioreactor samples; this could be related to faster degradation of the scaffolds under mechanical stimulation (FIG. 4L). These results suggest that, when exposed to stretch and flexing, cell seeded scaffolds show greater resistance to deformation and can withstand greater strain than those without cells under the same conditions. This data suggested that cellular seeding could support the mechanical properties of scaffolds in both static and bioreactor conditions.

### Example 5 P4HB/GelMa Remains Functional Under Physiological Stress In Vivo

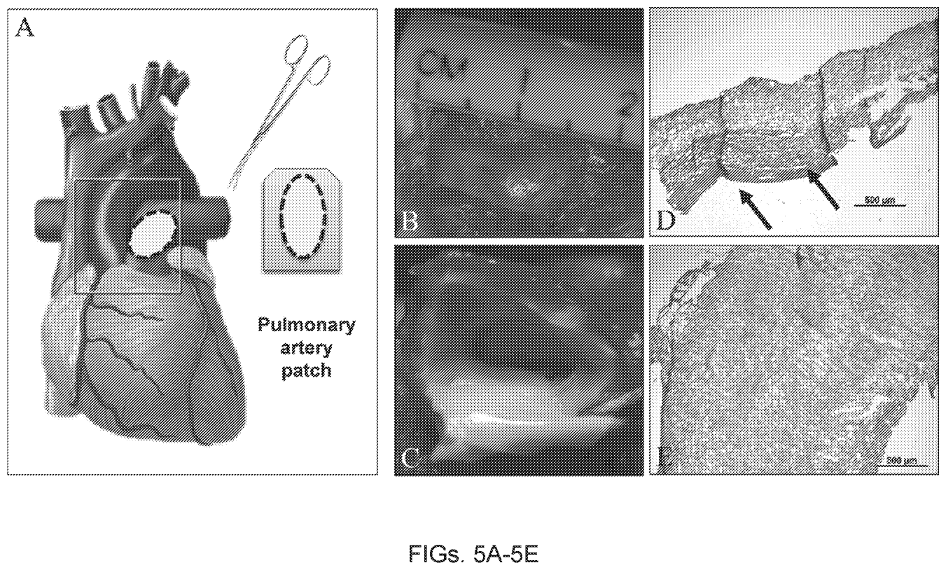

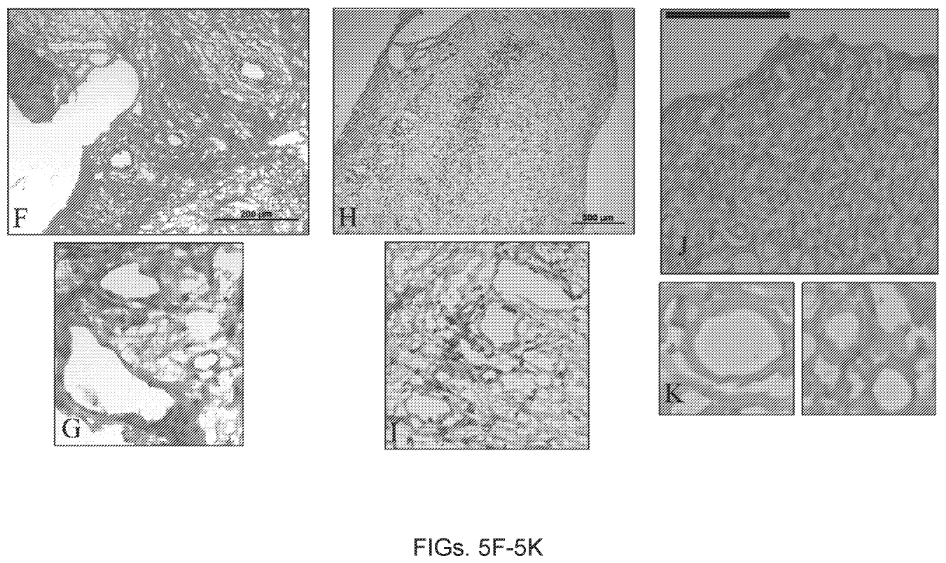

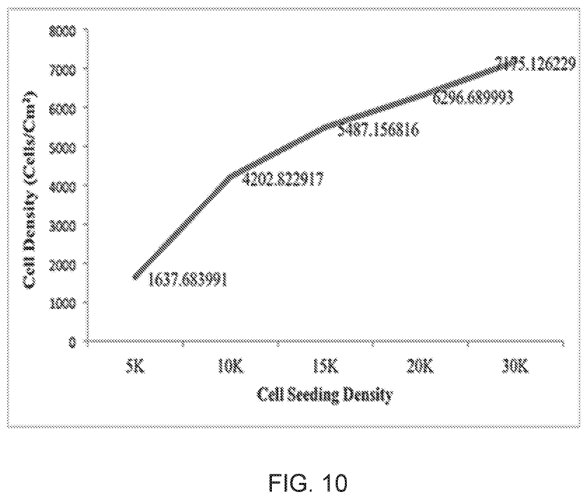

To evaluate the biocompatibility and functionality of the novel hybrid scaffold, we implanted P4HB/GelMa as a pulmonary artery patch in a sheep model previously described by our group (see Surgical Implantation section; FIG. 5A). FIG. 5B-C depicts the scaffolds—both sutured to the pulmonary artery and explanted—7 days post implantation. Autologous ovine MSCs and endothelial progenitor cells (EPCs) were used to seed the hybrid scaffold statically for 6 days prior to implantation. We previously conducted a thrombogenicity assay and determined that seeding the hybrid scaffold with autologous EPCs prevented thrombus formation (FIG. 10). In vivo, coating both surfaces of the hybrid scaffold with autologous EPCs also reduced the formation of surface thrombi (FIG. 5C-E). The explanted samples were cut and prepared for hematoxylin and eosin (H&E) staining on cross-sectional (D) and surface-oriented (E) cuts (FIG. 5D-E). H&E-stained sections showed tissue formation and cellularity throughout the scaffold. In addition, there was noticeable alignment of tissue matrix formed within the scaffold. Of note, this alignment occurred in the direction of blood flow (indicated by dark pink staining on FIG. 5D). Finally, the presence of myofibroblasts was confirmed by alpha-smooth muscle actin (α-SMA) staining (FIG. 5F, G). Myofibroblasts were seen abundantly within the center of the scaffolds, which indicated penetration and retention of cells. Collectively, this in vivo evaluation confirmed that our cell-seeded hybrid scaffold were non-thrombogenic and were capable of withstanding physiological pressures in the pulmonary artery. We have also demonstrated that leaflets constructed from this hybrid material were able to function well in a bioreactor system that mimics the stress, flexion, and shear experienced in the normal mammalian pulmonary circulation. The pulsatile cardiac bioreactor consists of a fluid loop placed into a system of elastic bladder, actuators, and sensors that manipulates the fluid loop and its contents to both create and monitor the same physiologic fluid dynamic conditions that a heart valve or vascular vessel would experience in-vivo.

Covering the trilayer P4HB composite scaffolds with EPC's showed that the cells formed a nice confluent monolayer that covered the whole construct after 96 hours of seeding by Calcein-AM live cell imaging. After 10 days of static culture in EBM-2 medium (Lonza, product code 190860), the scaffolds were placed in the bioreactor system and this whole system was kept in an incubator (37° C.). The flow in the bioreactor was set at approximately the pulmonary pressure and cell viability was confirmed after 7 and 14 days by Calcein-AM images. After 14 days of culture, the leaflets had opened and closed approximately a million times. No signs of material failure at the suture side or ruptures of the leaflet itself were observed.

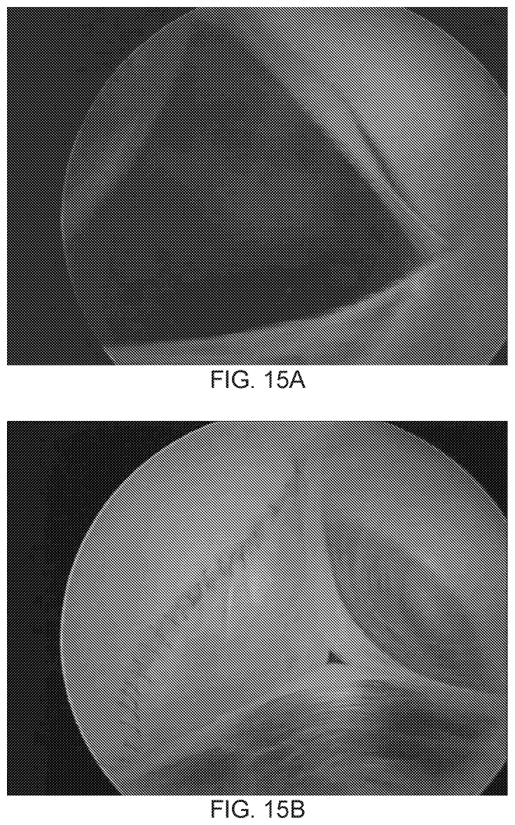

Further, an ex-vivo experiment was designed to test the trilayered P4HB composite scaffold (90-110 μm thickness) without cells was tested as a single leaflet replacement for pulmonary and aortic valve. Fresh hearts were obtained from a local slaughter house and the tri-layered scaffold was sutured through the top and bottom fibrous layers to the PA. The right ventricles were cannulated and connected to a water reservoir. The position of the fluid reservoir connected to the right ventricle (flow inlet) was chosen to provide a hydrostatic pressure similar to the systolic blood flow pressure at the position of the PV (about 30 mmHg) and the AV (about 80 mmHg). The pulmonary artery was connected to a second water reservoir through a tube, which provided 10 mmHg of pressure during diastole. Repetitive cycles of systole and diastole were manually generated by opening and closing the clamps attached to the inlet and outlet flow lines, with the implant visible during each cycle. The repetitive cycles of systole and diastole were manually controlled with the implant, visible during each cycle in real time. The scaffolds opening and closing was visualized using a surgical endoscope cannulated through the ventricles right beneath the PV position. Both, the PV and AV pressures were measured from the PV position. This ex-vivo test showed that under pulmonary pressure the valve moved in a similar fashion as the native leaflets and was able to fully enclose with the native valves (FIG. 14). Increasing the pressure comparable to aortic pressure showed the leaflet was still able to function properly without any failure of the material (FIG. 15). This indicates the scaffold could also be used as leaflet replacement for aortic valves.

### REFERENCES

[1] S. Brody, A. Pandit, Journal of biomedical materials research. Part B, Applied biomaterials 2007, 83, 16.
[2] J. P. Vacanti, R. Langer, The lancet 1999, 354, S32.
[3] D. W. Hutmacher, Biomaterials 2000, 21, 2529.
[4] M. S. Sacks, F. J. Schoen, J. E. Mayer, Annual review of biomedical engineering 2009, 11, 289.
[5] N. Annabi, S. M. Mithieux, P. Zorlutuna, G. Camci-Unal, A. S. Weiss, A. Khademhosseini, Biomaterials 2013, 34, 5496.
[6] N. Masoumi, N. Annabi, A. Assmann, B. L. Larson, J. Hjortnaes, N. Alemdar, M. Kharaziha, K. B. Manning, J. E. Mayer, A. Khademhosseini, Biomaterials 2014, 35, 7774.
[7] J. Rouwkema, N. C. Rivron, C. A. van Blitterswijk, Trends in biotechnology 2008, 26, 434.
[8] N. Masoumi, B. L. Larson, N. Annabi, M. Kharaziha, B. Zamanian, K. S. Shapero, A. T. Cubberley, G. Camci-Unal, K. Manning, J. E. Mayer, Advanced healthcare materials 2014, 3, 929.
[9] N. Masoumi, A. Jean, J. T. Zugates, K. L. Johnson, G. C. Engelmayr, Jr., Journal of biomedical materials research. Part A 2013, 101, 104.
[10] M. E. Kolewe, H. Park, C. Gray, X. Ye, R. Langer, L. E. Freed, Advanced materials 2013, 25, 4459.
[11] D. Gottlieb, T. Kunal, S. Emani, E. Aikawa, D. W. Brown, A. J. Powell, A. Nedder, G. C. Engelmayr, Jr., J. M. Melero-Martin, M. S. Sacks, J. E. Mayer, Jr., The Journal of thoracic and cardiovascular surgery 2010, 139, 723.
[12] M. Kharaziha, M. Nikkhah, S. R. Shin, N. Annabi, N. Masoumi, A. K. Gaharwar, G. Camci-Unal, A. Khademhosseini, Biomaterials 2013, 34, 6355.
[13] H. Niu, J. Mu, J. Zhang, P. Hu, P. Bo, Y. Wang, Journal of Materials Science: Materials in Medicine 2013, 24, 1535.
[14] R. Hashizume, Y. Hong, K. Takanari, K. L. Fujimoto, K. Tobita, W. R. Wagner, Biomaterials 2013, 34, 7353.
[15] J. L. Drury, D. J. Mooney, Biomaterials 2003, 24, 4337.
[16] R.-Z. Lin, Y.-C. Chen, R. Moreno-Luna, A. Khademhosseini, J. M. Melero-Martin, Biomaterials 2013, 34, 6785.
[17] P. S. Robinson, S. L. Johnson, M. C. Evans, V. H. Barocas, R. T. Tranquillo, Tissue Engineering Part A 2008, 14, 83.
[18] B. M. Baker, A. O. Gee, R. B. Metter, A. S. Nathan, R. A. Marklein, J. A. Burdick, R. L. Mauck, Biomaterials 2008, 29, 2348.
[19] M. Eslami, N. E. Vrana, P. Zorlutuna, S. Sant, S. Jung, N. Masoumi, R. A. Khavari-Nejad, G. Javadi, A. Khademhosseini, Journal of biomaterials applications 2014, 0885328214530589.
[20] J. J. Rice, M. M. Martino, L. De Laporte, F. Tortelli, P. S. Briquez, J. A. Hubbell, Advanced healthcare materials 2013, 2, 57.
[21] Y. C. Chen, R. Z. Lin, H. Qi, Y. Yang, H. Bae, J. M. Melero-Martin, A. Khademhosseini, Advanced functional materials 2012, 22, 2027.
[22] B. Duan, L. A. Hockaday, K. H. Kang, J. T. Butcher, Journal of biomedical materials research Part A 2013, 101, 1255; J. Hjortnaes, G. Camci-Unal, J. D. Hutcheson, S. M. Jung, F. J. Schoen, J. Kluin, E. Aikawa, A. Khademhosseini, Advanced healthcare materials 2015, 4, 121.
[23] D. P. Martin, S. F. Williams, Biochemical Engineering Journal 2003, 16, 97.
[24] N. Masoumi, M. C. Howell, K. L. Johnson, M. J. Niesslein, G. Gerber, G. C. Engelmayr Jr, Proceedings of the Institution of Mechanical Engineers, Part H: Journal of Engineering in Medicine 2014, 228, 576.
[25] L. N. Sierad, A. Simionescu, C. Albers, J. Chen, J. Maivelett, M. E. Tedder, J. Liao, D. T. Simionescu, Cardiovascular engineering and technology 2010, 1, 138; R. T. Tranquillo, Annals of the New York Academy of Sciences 2002, 961, 251; B.-S. Kim, J. Nikolovski, J. Bonadio, D. J. Mooney, Nature biotechnology 1999, 17, 979.
[26] M. S. Sacks, W. David Merryman, D. E. Schmidt, Journal of biomechanics 2009, 42, 1804; B. S. Frank, P. B. Toth, W. K. Wells, C. R. McFall, M. L. Cromwell, S. L. Hilbert, G. K. Lofland, R. A. Hopkins, Journal of Surgical Research 2012, 174, 39.
[27] T. Courtney, M. S. Sacks, J. Stankus, J. Guan, W. R. Wagner, Biomaterials 2006, 27, 3631.
[28] G. C. Engelmayr, M. Cheng, C. J. Bettinger, J. T. Borenstein, R. Langer, L. E. Freed, Nature materials 2008, 7, 1003.
[29] M. S. Sacks, A. P. Yoganathan, Philosophical transactions of the Royal Society of London. Series B, Biological sciences 2007, 362, 1369.
[30] N. Masoumi, K. L. Johnson, M. C. Howell, G. C. Engelmayr, Jr., Acta biomaterialia 2013, 9, 5974.
[31] J. A. Stella, J. Liao, Y. Hong, W. D. Merryman, W. R. Wagner, M. S. Sacks, Biomaterials 2008, 29, 3228.
[32] Y. Hong, J. Guan, K. L. Fujimoto, R. Hashizume, A. L. Pelinescu, W. R. Wagner, Biomaterials 2010, 31, 4249.
[33] N. Masoumi, N. Annabi, A. Assmann, B. L. Larson, J. Hjortnaes, N. Alemdar, M. Kharaziha, K. B. Manning, J. E. Mayer, Jr., A. Khademhosseini, Biomaterials 2014, 35, 7774.
[34] R. T. Tran, P. Thevenot, Y. Zhang, D. Gyawali, L. Tang, J. Yang, Materials 2010, 3, 1375.
[35] G. Sommer, M. Schwarz, M. Kutschera, R. Kresnik, P. Regitnig, A. Schriefl, H. Wolinski, S. Kohlwein, G. A. Holzapfel, Biomedical Engineering/Biomedizinische Technik 2013.
[36] A. Duprey, K. Khanafer, M. Schlicht, S. Avril, D. Williams, R. Berguer, European Journal of Vascular and Endovascular Surgery 2010, 39, 700.
[37] S. R. MacEwan, A. Chilkoti, Peptide Science 2010, 94, 60.
[38] Y. N. Zhang, R. K. Avery, Q. Vallmajo-Martin, A. Assmann, A. Vegh, A. Memic, B. D. Olsen, N. Annabi, A. Khademhosseini, Advanced functional materials 2015, 25, 4814.
[39] J. Raphel, A. Parisi-Amon, S. C. Heilshom, Journal of materials chemistry 2012, 22, 19429; C. Chou, R. Uprety, L. Davis, J. W. Chin, A. Deiters, Chemical Science 2011, 2, 480.
[40] J. A. Benton, C. A. DeForest, V. Vivekanandan, K. S. Anseth, Tissue Engineering Part A 2009, 15, 3221.
[41] G. C. Engelmayr Jr, L. Soletti, S. C. Vigmostad, S. G. Budilarto, W. J. Federspiel, K. B. Chandran, D. A. Vorp, M. S. Sacks, Annals of biomedical engineering 2008, 36, 700.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. An elastomeric scaffold for soft tissue engineering comprising a poly-4-hydroxybutyrate (P4HB) matrix.

2. The scaffold of claim 1, further comprising a hydrogel, preferably a photocrosslinkable hydrogel.

3. The scaffold of claim 2, wherein the photocrosslinkable hydrogel is gelatin or methacrylated gelatin (GelMa).

4. The scaffold of claim 2, comprising a P4HB matrix, wherein the hydrogel is distributed throughout the matrix.

5. The scaffold of claim 2, comprising an inner layer of a gelatin/P4HB composite, and an outer layer of P4HB on either side of the inner layer.

6. The elastomeric scaffold of claim 1, which is fabricated by dry spinning to generate aligned fibers of P4HB.

7. The elastomeric scaffold of claim 1, wherein the P4HB matrix has an average fiber diameter of 5-20 μm, preferably 8-10 μm.

8. The elastomeric scaffold of claim 1, which has a porosity of 10-15 μm.

9. The scaffold of claim 2, wherein the hydrogel encapsulates a plurality of cells, preferably stem cells, preferably mesenchymal stem cells (MSCs) or Valvular Interestitial Cells.

10. The scaffold of claim 9, wherein the surface of the scaffold comprises cells, preferably cells of a second cell type, preferably endothelial progenitor cells (EPCs), preferably derived from circulating blood.

11. A method of forming an artificial tissue, comprising culturing the scaffold of claim 10 in a cyclic stretch/flexure bioreactor or in a bioreactor that delivers flow, flexion, and shear signals to the scaffold.

12. An artificial tissue formed by the method of claim 11.

13. An artificial tissue formed by the method of claim 11, wherein the tissue is a heart valve leaflet, vascular conduit or blood vessel, or a portion thereof.

14. A method of replacing a tissue in a subject, the method comprising implanting into the subject the scaffold of claim 1.

15. A method of replacing a tissue in a subject, the method comprising implanting into the subject the tissue of claim 12.

16. A method of replacing a heart valve leaflet, vascular conduit or blood vessel, or a portion thereof, in a subject, the method comprising implanting into the subject the heart valve leaflet, vascular conduit or blood vessel of claim 13.

17. A method of forming an artificial tissue, the method comprising:
fabricating or providing an elastomeric scaffold comprising poly-4-hydroxybutyrate (P4HB), wherein the scaffold is fabricated by dry spinning to generate aligned fibers of P4HB to form an anisotropic matrix;
contacting the elastomeric scaffold with a hydrogel, preferably a photocrosslinkable hydrogel, wherein the hydrogel encapsulates a first plurality of cells, preferably stem cells, preferably mesenchymal stem cells (MSCs), under conditions such that the hydrogel is distributed throughout the scaffold;
optionally seeding the surface of the hydrogel-scaffold with a second plurality of cells, preferably cells of a different origin from the first plurality, preferably EPCs, preferably isolated from circulating blood;
exposing the cell-seeded scaffold to light sufficient to crosslink the hydrogel; and
culturing the scaffold under conditions sufficient to allow proliferation and optionally differentiation of the cells, thereby forming an artificial tissue.

18. The method of claim 17, wherein the artificial tissue is shaped to be used as a heart valve leaflet, vascular conduit or blood vessel.

19. The method of claim 17, wherein the photocrosslinkable hydrogel is methacrylated gelatin (GelMa).

20. A method of forming an artificial tissue, the method comprising:
fabricating or providing an elastomeric scaffold comprising a poly-4-hydroxybutyrate (P4HB)/gelatin matrix comprising an inner layer of a gelatin/P4HB composite, and an outer layer of P4HB on either side of the inner layer, wherein the scaffold is fabricated by:
generating a first layer of aligned fibers of P4HB;
forming a layer comprising a P4HB/gelatin composite on the matrix; and
generating a second layer of aligned fibers of P4HB;
preferably wherein the gelatin encapsulates a first plurality of cells, preferably stem cells, preferably mesenchymal stem cells (MSCs);
optionally seeding the surface of the hydrogel-scaffold with a second plurality of cells, preferably cells of a different origin from the first plurality, preferably EPCs, preferably isolated from circulating blood;
exposing the cell-seeded scaffold to light sufficient to crosslink the hydrogel; and
culturing the scaffold under conditions sufficient to allow proliferation and optionally differentiation of the cells, optionally comprising culturing the scaffold of claim 10 in a cyclic stretch/flexure bioreactor or in a bioreactor that delivers flow, flexion, and shear signals to the scaffold,
thereby forming an artificial tissue.

21. A method of replacing a tissue in a subject, the method comprising implanting into the subject the tissue of claim 20.

22. A method of replacing a heart valve leaflet, vascular conduit or blood vessel, or a portion thereof, in a subject, the method comprising implanting into the subject the heart valve leaflet, vascular conduit or blood vessel of claim 22.
